(19) United States
(12) Patent Application Publication
(10) Pub. No.: US 2013/0123108 A1
(43) Pub. Date: May 16, 2013

(54) ACTIVE SUBSTANCE COMPOSITION COMPRISING AT LEAST ONE NITROGEN ATOM-CONTAINING, HYPERBRANCHED POLYMER

(71) Applicant: BASF SE, Ludwigshafen (DE)

(72) Inventors: Nathalie Bouillo, Baden-Baden (DE); Christian Krüger, Saulheim (DE); Marianna Pierobon, Ludwigshafen (DE); Bernd Bruchmann, Freinsheim (DE); Jean-Francois Stumbe, Strasbourg (FR); Ronald Frans Maria Lange, Gumlingen (CH)

(73) Assignee: BASF SE, Ludwigshafen (DE)

(21) Appl. No.: 13/670,965

(22) Filed: Nov. 7, 2012

Related U.S. Application Data
(62) Division of application No. 11/816,723, filed on Aug. 21, 2007, now abandoned.

(30) Foreign Application Priority Data
Feb. 21, 2005 (DE) 102005007844.3

(51) Int. Cl. A61K 47/34; A61K 8/85; A61K 8/88; A01N 25/22; A61K 8/87

(52) U.S. Cl. CPC A61K 47/34; A01N 25/22; A61K 8/87; A61K 8/88; A61K 8/85
USPC 504/360; 514/772.3; 514/788; 504/362

(57) ABSTRACT

Compositions comprising: (a) at least one hyperbranched polymer comprising nitrogen atoms; and (b) at least one substance exhibiting a solubility in water at 25° C. and 1013 mbar of less than 10 g/l are disclosed along with methods of preparing such compositions.

### CROSS-REFERENCE TO RELATED APPLICATIONS

This Application is a Division of application Ser. No. 11/816,723 filed on Aug. 21, 2007. application Ser. No. 11/816,723 is a national stage application, under 35 U.S.C. §371, of PCT/EP2006/001515, filed Feb. 20, 2006, which claims priority of German Application No. 10 2005 007 844.3, filed Feb. 21, 2005, the entirety of all of which are incorporated herein by reference.

### BACKGROUND OF THE INVENTION

Active substances for pharmaceuticals, plant protection, cosmetics and material protection, i.e. substances which, even in low concentration, already exhibit an activity, e.g. a pharmacological activity in an organism, a physiological activity in a plant or a harmful organism, a cosmetic activity, and the like, are frequently formulated and used in the form of aqueous active substance preparations. Alternatively, it is also possible to formulate and administer in solid form, e.g. as powder or pressed article (tablet, and the like), the transportation to the actual site of action, however, comprising the conversion to an aqueous form.

The main problem with aqueous active substance preparations is the low solubility in water of many active substances, which frequently amounts to less than 5 g/l at 23° C./1013 mbar. Aqueous formulations of such active substances can exist as heterogeneous systems in which the active substance is present as emulsified or dispersed phase in a continuous aqueous phase. Emulsifiers or dispersants are usually introduced in order to stabilize these per se metastable systems. However, their stabilizing effect is frequently unsatisfactory, so that separation of the active substance, for example creaming or sedimentation of the active substance, can occur, in particular if the aqueous formulation is stored for a relatively long time at high temperature and/or at highly changeable temperatures or in the vicinity of the freezing point. This problem is then particularly pronounced if the active substance has a tendency to crystallize. Furthermore, in many cases, a solubilization, i.e. an improvement in solubility through surface-active compounds, is striven for, which transforms the sparingly water-soluble or water-insoluble substances into clear, highly opalescent, aqueous solutions without, in this connection, the chemical structure of these substances undergoing a change. These solubilizates are characterized in that the sparingly water-soluble or water-insoluble substance is present dissolved in the molecular assemblies of the surface-active compounds, which are formed in aqueous solution. The resulting solutions are stable single-phase systems which appear optically clear to opalescent and can be prepared without introducing energy. Solubilizers can, for example, improve the appearance of cosmetic formulations and of edible preparations by making the formulations transparent. In addition, in the case of pharmaceutical preparations, the bioavailability and therefore the activity of pharmaceuticals can also be increased by the use of solubilizers.

Organic solvents are also frequently used for the preparation of aqueous formulations of water-insoluble active substances. Thus, water-miscible solvents are frequently used as solvating agents, i.e. for increasing the solubility of the active substance in the aqueous phase. In turn, water-immiscible solvents are used to convey into a liquid phase an active substance which is solid at the temperature of use, which liquid phase can then be emulsified. In contrast to the solid active substance, the active substance is dissolved at the molecular level in the emulsion via the solvent and on application is more readily available and more effective. However, the use of organic solvents is undesirable, on the basis of the well-known VOC problem, for reasons of health and safety at work, environmental aspects and partly also toxicological reasons.

The formulation of water-insoluble active substances in the form of aqueous micro- or nanoemulsions has been proposed on several occasions. However, comparatively large amounts of emulsifier and of organic solvents are necessary for the preparation of such micro- or nanoemulsions. However, the high proportion of emulsifiers is not only a cost factor but can also lead to problems when the formulations are used. In turn, solvents are also undesirable for health and safety at work reasons and for cost reasons. An additional problem of such microemulsions is their instability with regard to separation.

Furthermore, aqueous polymer/active substance preparations obtained by radical aqueous emulsion polymerization of a monomer emulsion, in which the active substance is present in the monomer droplets of the monomer emulsion to be polymerized, have been described on several occasions. However, this process is restricted to those active substances which are readily soluble in the monomers. As a rule, they are substances which are liquid at ambient temperature.

The use of amphiphilic copolymers to dissolve water-insoluble active substances in an aqueous vehicle has also been proposed on several occasions. Thus, for example, US 2003/0009004 proposes, for this purpose, amphiphilic block copolymers which comprise a hydrophilic polyethyleneimine block and a hydrophobic block of a biodegradable aliphatic polyester.

US 2003/0157170 discloses anhydrous active substance compositions comprising an amphiphilic diblock copolymer with a polyester as hydrophobic constituent and an additive. The compositions form, on diluting with water, micelles which comprise active substance.

WO 02/82900 discloses the use of amphiphilic block copolymers to prepare aqueous suspensions of water-insoluble plant protection active substances. The block copolymers used can be obtained by “living” or “controlled” radical block copolymerization of ethylenically unsaturated monomers.

U.S. Pat. No. 4,888,389 discloses block copolymers exhibiting a polyisobutene block and a hydrophilic block, for example a polyether block.

The unpublished German patent application 10 2004 027 835.0 discloses the use of amphiphilic polymer compositions, exhibiting blocks of hydrophilic and hydrophobic polymers linked by reaction with polyisocyanates, to prepare aqueous formulations of active substances and effect substances which are insoluble or only to a small extent soluble in water.

Random amphiphilic copolymers have also been used as solubilizers. Thus, EP-A 0 876 819 relates to the use of copolymers of N-vinylpyrrolidone and alkylacrylic acids as solubilizers.

EP-A 0 953 347 relates to the use of graft polymers comprising polyalkylene oxide as solubilizers.

The use of polymerized fatty acid derivatives and fatty alcohol derivatives as solubilizers is known from EP-A 0 943 340.

EP-A 0 948 957 discloses the use of copolymers of monoethylenically unsaturated carboxylic acids as solubilizers.

R. Haag discloses, in Angew. Chemie, 2004, 116, pp. 280-284, supramolecular active substance transportation systems based on polymeric core-shell architectures. Dendritic core-shell architectures, based on highly branched polyglycerol and poly(ethyleneimine), are also disclosed in this connection.

The unpublished German patent application 10 2004 037 850.9 discloses aqueous compositions of active substances which are insoluble or only to a small extent soluble in water obtained by carrying out, in the presence of an aqueous suspension of the active substance particle, a first emulsion polymerization with an aqueous dispersion of polymer/active substance particles being obtained and by subsequently subjecting this to a second emulsion polymerization in the presence of at least one neutral monoethylenically unsaturated monomer.

The use of hyperbranched polymers to prepare aqueous active substance compositions of sparingly water-soluble active substances is not disclosed in the abovementioned documents. However, the use of such polymers for a multitude of different intended purposes is known. Thus, for example, WO 2004/037881 discloses substrates comprising on their surfaces at least one hyperbranched polymer exhibiting urethane and/or urea groups. WO 2004/094505 discloses stabilizers, which protect plastics from damage due to heat, UV radiation, and the like, which are covalently bonded via an anchoring group to a highly branched polymer.

### BRIEF SUMMARY OF THE INVENTION

The present invention relates to an active substance or effect substance composition comprising at least one active substance or effect substance which is sparingly soluble in water and at least one hyperbranched polymer comprising nitrogen atoms.

It is therefore an object of the invention to prepare preparations of water-insoluble or sparingly water-soluble active substances, in particular of active substances for pharmaceuticals, cosmetics, plant protection or material protection. These active substance compositions should be easy to prepare and should exhibit no or only a very small content of volatile organic substances. Furthermore, high stability of the resulting aqueous active substance compositions with regard to separation events on lengthy storage and on diluting with water is desirable.

Surprisingly, we have found that this object is achieved by the use of hyperbranched polymers comprising nitrogen atoms as solubilizers.

The present invention consequently relates to an active substance or effect substance composition comprising
A) at least one hyperbranched polymer comprising nitrogen atoms, and
B) at least one active substance or effect substance exhibiting a solubility in water at 25° C. and 1013 mbar of less than 10 g/l.

### DETAILED DESCRIPTION OF THE INVENTION

The hyperbranched polymers introduced according to the invention are advantageously suitable for the stabilization of water-insoluble (or only water-soluble to a small extent) active substances and effect substances in the aqueous phase and consequently make possible the preparation of aqueous formulations of such active substances and effect substances. They are also suitable for the preparation of solid formulations of these active substances and effect substances, which can be converted to an aqueous formulation, e.g. as commercial, administration or active form. This can also be carried out even after application of the solid composition (e.g. in the digestive tract of an organism and the like).

The “solubility improvement” targeted with the polymers used according to the invention is consequently understood in the broad sense in the context of the present invention. It includes, first, the stabilization of heterogeneous systems in which the active substance is present as emulsified or disperse phase in an aqueous medium as continuous phase. It includes, furthermore, the stabilization of transitional stages to homogeneous solutions, such as colloidal solutions, and the like, up to molecularly disperse solutions. It also includes a solubility improvement in the sense of a solubilization in which the poorly water-soluble or water-insoluble substances are converted to clear, highly opalescent, aqueous solutions. Finally, it also includes the capability of forming “solid solutions”.

A low (poor) solubility represents in the context of this invention, a solubility of the active substance or effect substance in water of less than 10 g/l, in particular of less than 1 g/l and especially of less than 0.1 WI, at 25° C. and 1013 mbar.

The aqueous active substance compositions of water-insoluble active substances or effect substances prepared using the hyperbranched polymers comprising nitrogen atoms comprise, in addition to an aqueous medium as continuous phase, at least one active substance and/or effect substance solubilized or dispersed in the continuous phase which exhibits a solubility in water at 25° C./1013 mbar of less than 10 g/l, in particular of less than 1 g/l and especially of less than 0.1 g/l, as well as at least one hyperbranched polymer comprising nitrogen atoms.

The active substance is present in the continuous aqueous phase in an extremely finely divided form. This can, for example, be put down to the fact that the active substance forms aggregates in the aqueous phase with the polymers A). These aggregates as a rule exhibit mean particle sizes of less than 1 μm, frequently of less than 500 nm, in particular of less than 400 nm, especially of less than 300 nm. Depending on the kind of polymer and of active substance or effect substance, and also depending on the concentration ratios, the aggregates can even become so small that they are no longer present in the form of detectable discrete particles but are present in the dissolved form (particle size <10 nm).

The particle sizes given here are weight-average particle sizes, and they can be determined by dynamic light scattering. Methods for this are familiar to a person skilled in the art, for example from H. Wiese in D. Distler, Wässrige Polymerdispersionen [Aqueous Polymer Dispersions], Wiley-VCH 1999, chapter 4.2.1, p. 40ff, and the literature cited therein, as well as H. Auweter, D. Horn, J. Colloid Interf. Sci., 105 (1985), 399, D. Lilge and D. Horn, Colloid Polym. Sci., 269 (1991), 704, or H. Wiese and D. Horn, J. Chem. Phys., 94 (1991), 6429.

The terms “aqueous medium” and “aqueous phase” comprise, here and subsequently, water, aqueous mixtures of water with up to 10% by weight, based on the mixture, of organic solvents which are miscible with water, and solutions of solids in water or in the aqueous mixtures. Examples of water-miscible solvents comprise C₃-C₄ ketones, such as acetone and methyl ethyl ketone, cyclic ethers, such as dioxane and tetrahydrofuran, C₁-C₄ alkanols, such as methanol, ethanol, n-propanol, isopropanol, n-butanol or tert-butanol, polyols and their mono- and dimethyl ethers, such as glycol, propanediol, ethylene glycol monomethyl ether, diethylene glycol, diethylene glycol monomethyl ether, diethylene glycol dimethyl ether or glycerol, furthermore C₂-C₃ nitriles, such as acetonitrile and propionitrile, dimethyl sulfoxide, dimethylformamide, formamide, acetamide, dimethylacetamide, butyrolactone, 2-pyrrolidone and N-methylpyrrolidone.

The term “functionality” represents, here and subsequently, the average number of the respective functional groups per molecule or per polymer chain.

An advantage of the active substance compositions according to the invention is that they can also be formulated low in solvents (content of volatile solvents <10% by weight, based on the weight of the active substance composition) or even free from solvents (content of volatile solvents <1% by weight, based on the weight of the active substance composition).

A further advantage is to be seen in that the aqueous active substance compositions according to the invention can as a rule be dried to a redispersible powder. That is, by removal of the aqueous phase during the drying, a finely divided powder is obtained which can, without any bother, be dissolved or dispersed in water without the occurrence of a significant increase in particle size.

In the context of the present invention, the term “hyperbranched polymers” comprises very generally polymers which are distinguished by a branched structure and a high functionality. Reference may also be made, for the general definition of hyperbranched polymers, to P. J. Flory, J. Am. Chem. Soc., 1952, 74, 2718, and H. Frey et al., Chem. Eur. J., 2000, 6, No. 14, 2499. The “hyperbranched polymers” within the meaning of the invention include star polymers, dendrimers and high molecular weight polymers different therefrom, such as, e.g., comb polymers. “Star polymers” are polymers in which three or more chains start at one center. The center can in this connection be an individual atom or a group of atoms. “Dendrimers” (hyperbranched polymers, cascade polymers, arborols (=dendrimers with hydroxyl groups), isotropically branched polymers, isobranched polymers, starburst polymers) are molecularly uniform macromolecules with a highly symmetrical structure. Dendrimers are derived structurally from the star polymers in which the individual chains are each for their part branched in a star-like way. They arise starting from small molecules through a continually repeating reaction sequence, resulting in ever higher branchings, at the ends of which are each time found functional groups which are in turn starting points for further branchings. Thus, the number of the monomer end groups grows exponentially with each reaction step, resulting at the end in a spherical tree structure. A characteristic feature of the dendrimers is the number of the reaction stages (generations) carried out to construct them. Due to their uniform structure, dendrimers as a rule exhibit a defined molar mass.

Both molecularly and structurally nonuniform hyperbranched polymers exhibiting side chains of varying length and branching, as well as a molar mass distribution, are preferably suitable.

“ABₓ monomers” are particularly suitable for the synthesis of these hyperbranched polymers. These AB, monomers exhibit two different functional groups A and B which can react with one another for the formation of a linkage. The functional group A is in this connection only present once per molecule and the functional group B twice or several times. Reaction of said ABₓ monomers with one another produces essentially noncrosslinked polymers with regularly arranged branching positions. The polymers exhibit almost exclusively B groups at the chain ends. Further details will be found, for example, in Journal of Molecular Science, Rev. Macromol. Chem. Phys., C37(3), 555-579 (1997).

The hyperbranched polymers used according to the invention preferably exhibit a degree of branching (DB) corresponding to an average number of dendritic linkages and terminal units per molecule of 10 to 100%, preferably 10 to 90% and in particular 10 to 80%. Reference may be made, for the definition of the “Degree of Branching”, to H. Frey et al., Acta Polym., 1997, 48, 30.

Hyperbranched polymers, i.e. molecularly and structurally nonuniform polymers, are preferably used. These are as a rule simple and consequently more economical to prepare than dendrimers. However, of course, structurally and molecularly uniform dendrimeric polymers and star polymers can also be used to obtain an advantageous surface modification.

The hyperbranched polymers A) comprising nitrogen atoms are preferably chosen from polyurethanes, polyureas, polyamides, polyesteramides, polyesteramines and blends thereof.

The hyperbranched polymers used according to the invention preferably exhibit, in addition to the groups resulting from the synthesis of the hyperbranched structure (e.g. in the case of hyperbranched polyurethanes, urethane and/or urea groups or additional groups resulting from the reaction of isocyanate groups; in the case of hyperbranched polyamides, amide groups, and the like), at least four additional functional groups. The maximum number of these functional groups is as a rule not critical. However, in many cases, it is not more than 100. The amount of functional groups is preferably 4 to 100, especially 5 to 30 and more especially 6 to 20.

Preference is given to polymers which exhibit a weight-average molecular weight in the range of approximately 500 to 100 000, preferably 750 to 50 000, in particular 1000 to 30 000.

In the context of the present invention, the expression “alkyl” comprises straight-chain and branched alkyl groups. Suitable short-chain alkyl groups are, e.g., straight-chain or branched C₁-C₇alkyl, preferably C₁-C₆alkyl and particularly preferably C₁-C₄alkyl groups. These include in particular methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl, and the like. Suitable long-chain C₈-C₃₀ alkyl or C₆-C₃₀alkenyl groups are straight-chain and branched alkyl or alkenyl groups. In this connection, they are preferably mainly linear alkyl residues, such as those also present in natural or synthetic fatty acids and fatty alcohols and also oxo alcohols, which, if appropriate, in addition can be mono-, di- or polyunsaturated. These include, e.g., n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene), and the like.

The expression “alkylene” within the meaning of the present invention represents straight-chain or branched alkanediyl groups with 1 to 4 carbon atoms, e.g. methylene, 1,2-ethylene, 1,3-propylene, and the like.

Cycloalkyl preferably represents C₅-C₈ cycloalkyl, such as cyclopentyl, cyclohexyl, cycloheptyl or cyclooctyl.

Aryl comprises unsubstituted and substituted aryl groups and preferably represents phenyl, tolyl, xylyl, mesityl, naphthyl, fluorenyl, anthracenyl, phenanthrenyl, naphthacenyl and in particular phenyl, tolyl, xylyl or mesityl.

### I) Hyperbranched Polyurethanes

In a first embodiment, the aqueous active substance composition according to the invention comprises at least one hyperbranched polyurethane polymer.

The term “polyurethanes” comprises, in the context of this invention, not only those polymers whose repeat units are bonded to one another via urethane groups but very generally polymers which can be obtained by reaction of at least one di- and/or polyisocyanate with at least one compound exhibiting at least one group which is reactive with regard to isocyanate groups. These include polymers whose repeat units, in addition to urethane groups, are also bonded by urea, allophanate, biuret, carbodiimide, amide, uretonimine, uretdione, isocyanurate or oxazolidone (oxazolidinone) groups (see, for example, Plastics Handbook, Saechtling, 26th edition, p. 491ff, Carl Hanser Verlag, Munich, 1995). The term “polyurethanes” comprises in particular polymers exhibiting urethane and/or urea groups.

The hyperbranched polymers used according to the invention preferably exhibit, in addition to urethane and/or urea groups (or additional groups resulting from the reaction of isocyanate groups), at least four additional functional groups. The amount of functional groups is preferably 4 to 100, particularly preferably 5 to 30 and in particular 6 to 20.

Preference is given to polyurethanes exhibiting a weight-average molecular weight in the range of approximately 500 to 100 000, preferably 1000 to 50 000.

Their content of urethane and/or urea groups (and, if present, additional groups obtained by reaction of an isocyanate group with a correspondingly reactive group with an active hydrogen atom) preferably lies in a range of 0.5 to 10 mol/kg, particularly preferably 1 to 10 mol/kg, especially 2 to 8 mol/kg.

The synthesis of hyperbranched polyurethanes and polyureas which can be used in accordance with the invention can, for example, be carried out as described below.

Use is preferably made, in the synthesis of the hyperbranched polyurethanes and polyureas, of ABₓ monomers exhibiting both isocyanate groups and groups which can react with isocyanate groups for the formation of a linkage. x is a natural number between 2 and 8. x is preferably 2 or 3. Either A relates to the isocyanate groups and B relates to groups which react with them or the opposite case may exist.

The groups which react with isocyanate groups are preferably OH, NH₂, NRH, SH or COOH groups.

The ABₓ monomers can be prepared in a known way using various techniques.

ABₓ monomers can, for example, be synthesized according to the method disclosed in WO 97/02304 with the use of protective group techniques. By way of example, this technique was illustrated by the preparation of an AB₂ monomer from 2,4-toluoylene diisocyanate (TDI) and trimethylolpropane. First, one of the isocyanate groups of the TDI is blocked in a known way, for example by reaction with an oxime. The remaining free NCO group is reacted with trimethylolpropane, one of the three OH groups reacting with the isocyanate group. After cleavage of the protective group, a molecule with one isocyanate group and 20H groups is obtained.

In a particularly advantageous way, the ABₓ molecules can be synthesized according to the method disclosed in DE-A 199 04 444, in which no protective groups are required. In this method, di- or polyisocyanates are used and are reacted with compounds exhibiting at least two groups which react with isocyanate groups. At least one of the reaction partners exhibits groups with varying reactivity with regard to the other reaction partner. Preferably, both reaction partners exhibit groups with varying reactivity with regard to the other reaction partner. The reaction conditions are chosen so that only certain reactive groups can react with one another.

In addition, ABₓ molecules can be prepared as disclosed in the German patent application P 102 04 979.3. In this instance, isocyanate groups protected by blocking agents are reacted with polyamines to give polyureas.

Possible di- or polyisocyanates are the aliphatic, cycloaliphatic, araliphatic and aromatic di- or polyisocyanates known as state of the art and mentioned subsequently by way of example. Mention may preferably be made, in this connection, of 4,4′-diphenylmethane diisocyanate, the mixtures of monomeric diphenylmethane di-isocyanates and oligomeric diphenylmethane diisocyanates (polymer MDI), tetramethylene diisocyanate, tetramethylene diisocyanate trimers, hexamethylene diisocyanate, hexamethylene diisocyanate trimers, isophorone diisocyanate trimer, 4,4′-methylenebiscyclohexane diisocyanate, xylylene diisocyanate, tetramethylxylylene diisocyanate, dodecane diisocyanate, lysine alkyl ester diisocyanate, alkyl representing C₁-C₁₀ alkyl, 1,4-diisocyanatocyclohexane or 4-isocyanatomethyl-1,8-octamethylene diisocyanate.

Di- or polyisocyanates exhibiting NCO groups of varying reactivity are suitable particularly preferably for the synthesis of polyurethanes and polyureas. Mention may be made in this connection of 2,4-toluoylene diisocyanate (2,4-TDI), 2,4′-diphenylmethane diisocyanate (2,4′-MD1), triisocyanatotoluene, isophorone diisocyanate (IPDI), 2-butyl-2-ethylpentamethylene diisocyanate, 2,2,4- or 2,4,4-trimethyl-1,6-hexamethylene diisocyanate, 2-isocyanatopropylcyclohexane isocyanate, 3(4)-isocyanatomethyl-1-methylcyclohexane isocyanate, 1,4-diisocyanato-4-methylpentane, 2,4′-methylenebiscyclohexane diisocyanate and 4-methylcyclohexane 1,3-diisocyanate (H-TDI).

Isocyanates, the NCO groups of which are first equally reactive, in which, however, the first addition of a reactant to an NCO group can induce a fall in reactivity in the second NCO group, are furthermore suitable for the synthesis of the polyurethanes and polyureas. Examples thereof are isocyanates, the NCO groups of which are coupled via a delocalized p-electron system, e.g. 1,3- and 1,4-phenylene diisocyanate, 1,5-naphthalene diisocyanate, biphenyl diisocyanate, tolidine diisocyanate or 2,6-toluoylene diisocyanate.

Furthermore, use may be made, for example, of oligo- or polyisocyanates which can be prepared from the abovementioned di- or polyisocyanates or mixtures thereof by linking by means of urethane, allophanate, urea, biuret, uretdione, amide, isocyanurate, carbodiimide, uretonimine, oxadiazinetrione or iminooxadiazinedione structures.

Use is preferably made, as compounds with at least two groups which are reactive with isocyanates, of di-, tri- or tetrafunctional compounds, the functional groups of which exhibit a varying reactivity with regard to NCO groups.

Preference is given, for the preparation of polyurethanes and polyurea-polyurethanes, to compounds with at least one primary and at least one secondary hydroxyl group, at least one hydroxyl group and at least one mercapto group, particularly preferably with at least one hydroxyl group and at least one amino group in the molecule, in particular aminoalcohols, aminodiols and aminotriols, since the reactivity of the amino group is clearly higher in comparison with the hydroxyl group in the reaction with isocyanate.

Examples of the abovementioned compounds with at least two groups which react with isocyanates are propylene glycol, glycerol, mercaptoethanol, ethanolamine, N-methylethanolamine, diethanolamine, ethanolpropanolamine, dipropanolamine, di-isopropanolamine, 2-amino-1,3-propanediol, 2-amino-2-methyl-1,3-propanediol or tris(hydroxymethyl)aminomethane. Furthermore, mixtures of the above-mentioned compounds can also be used.

Isocyanate-reactive products exhibiting at least two amino groups in the molecule are preferably used for the preparation of polyureas.

These are, for example, ethylenediamine, N-alkylethylenediamine, propylenediamine, N-alkylpropylenediamine, hexamethylenediamine, N-alkylhexamethylenediamine, diaminodicyclohexylmethane, phenylenediamine, isophoronediamine, amine-terminated polyoxyalkylenepolyols (referred to as Jeffamines), bis(aminoethyl)amine, bis(aminopropyl)amine, bis(aminohexyl)amine, tris(aminoethyl)amine, tris(aminopropyl)amine, tris(aminohexyl)amine, trisaminohexane, 4-aminomethyl-1,8-octamethylenediamine, N′-(3-aminopropyl)-N,N-dimethyl-1,3-propanediamine, trisaminononane or melamine. Furthermore, mixtures of the above-mentioned compounds can also be used.

The preparation of an ABₓ molecule for the preparation of a polyurethane from a diisocyanate and an aminodiol is illustrated here by way of example. In this connection, first one mole of a diisocyanate is reacted with one mole of an aminodiol at low temperatures, preferably in the range between −10 and 30° C. In this temperature range, the urethane formation reaction is virtually completely suppressed and the NCO groups of the isocyanate react exclusively with the amino group of the aminodiol. The ABₓ molecule formed, in this instance an AB₂ type, exhibits a free NCO group and two free OH groups and can be used for the synthesis of a hyperbranched polyurethane.

This AB₂ molecule can react intermolecularly, by warming and/or addition of catalyst, to give a hyperbranched polyurethane. The synthesis of the hyperbranched polyurethane can advantageously take place at elevated temperature, preferably in the range between 30 and 80° C., without prior isolation of the AB₂ molecule in an additional reaction step. On using the AB₂ molecule with two OH groups and one NCO group described, a hyperbranched polymer is produced which, per molecule, exhibits one free NCO group and, depending on the degree of polymerization, a more or less large number of OH groups. The reaction can be carried out up to high conversions, through which very high molecular weight structures are obtained. However, it can also, for example, be terminated by addition of suitable monofunctional compounds or by addition of one of the starting compounds for the preparation of the AB₂ molecule on reaching the desired molecular weight. Depending on the starting compound used for the termination, either completely NCO-terminated or completely OH-terminated molecules are produced.

Alternatively, an AB₂ molecule can also be prepared, for example, from 1 mol of glycerol and 2 mol of 2,4-TDI. At low temperature, the primary alcohol groups and the isocyanate group in the 4-position preferably react and an adduct is formed which exhibits one OH group and two isocyanate groups and which, as described, can be converted at higher temperatures to give a hyperbranched polyurethane. There is produced first a hyperbranched polymer which exhibits one free OH group and, depending on the degree of polymerization, a more or less large number of NCO groups.

The preparation of the hyperbranched polyurethanes and polyureas can in principle be carried out without solvent but is preferably carried out in solution. All compounds liquid at the reaction temperature and inert with regard to the monomers and polymers are suitable in principle as solvent.

Other products are accessible by additional alternative synthetic forms. Mention may be made here, for example, of:

AB₃ molecules can, for example, be obtained by reaction of diisocyanates with compounds with at least 4 groups which are reactive with respect to isocyanates. Mention may be made, by way of example, of the reaction of toluoylene diisocyanate with tris(hydroxymethyl)aminomethane.

Polyfunctional compounds can also be used to terminate the polymerization, which compounds can react with the respective A groups. In this way, several small hyperbranched molecules can be linked together to give a large hyperbranched molecule.

Hyperbranched polyurethanes and polyureas with chain-extended branches can, for example, be obtained by additionally using, in the polymerization reaction, in addition to the ABₓ molecules, in the molar ratio 1:1, a diisocyanate and a compound which exhibits two groups which react with isocyanate groups. These additional AA or BB compounds can also even have available additional functional groups which, under the reaction conditions, may not, however, be reactive with regard to the A or B groups. In this way, additional functionalities can be introduced into the hyperbranched polymer. Additional suitable alternative synthetic forms for hyperbranched polymers are found in DE-A-100 13 187 and DE-A-100 30 869 and in the German patent applications P 103 51 401.5 and P 10 2004 006304.4

### II) Hyperbranched Polyamides

Hyperbranched polyamides are disclosed, for example, in U.S. Pat. No. 4,507,466, U.S. Pat. No. 6,541,600, U.S. Pat. No. 2,003,055209, U.S. Pat. No. 6,300,424, U.S. Pat. No. 5,514,764 and WO 92/08749, reference to which is made here in their entirety.

A suitable procedure for the preparation of hyperbranched polyamides starts out from polyfunctional amines and polycarboxylic acids, use being made of at least one polyfunctional compound exhibiting three or more than three (e.g. 4, 5, 6, and the like) functional groups. Formally, in this procedure, a first class of monomers with two identical functional groups A₂ (e.g., a dicarboxylic acid or a diamine) is then reacted with a second class of monomers Bₙ, this second class comprising at least one compound with more than equal functional groups (e.g., at least one tricarboxylic acid (n=3) or a higher than trivalent carboxylic acid or at least one triamine (n=3) or a higher than trivalent amine). Preferably, the second class of monomers comprises at least one divalent monomer B₂ which exhibits two functional groups complementary to the monomers A₂. Preferably, the monomers Bₙ exhibit a mean functionality of at least 2.1 (n=2.1). Preferably, for the preparation of hyperbranched polyamides according to this alternative form, the monomers A₂ are used in a molar excess with regard to the monomers Bₙ. Preferably, the molar ratio of monomers A₂ to monomers Bₙ lies in a range from 1:1 to 20:1, particularly preferably of 1.1:1 to 10:1, in particular 1.2:1 to 5:1. In a preferred embodiment, a hyperbranched prepolymer with terminal groups A is first prepared and is further reacted subsequently with at least one monomer B₂ and/or Bₙ. To prepare the prepolymer, use is preferably made of monomers A₂ and monomers Bₙ in a molar ratio of 1:1 to 20:1, particularly preferably of 1.1:1 to 10:1, especially 1.2:1 to 5:1.

An additional suitable procedure for the preparation of hyperbranched polyamides starts out from polyfunctional aminocarboxylic acids, use being made of at least one polyfunctional compound exhibiting three or more than three (e.g., 4, 5, 6 and the like) functional groups, i.e. what is referred to as an ABₓ monomer (x is greater than or equal to 2). These can then be reacted with additional monomers AB, A₂ and/or B₂.

Suitable dicarboxylic acids are, for example, oxalic acid, malonic acid, succinic acid, glutaric acid, adipic acid, pimelic acid, suberic acid, azelaic acid, sebacic acid, undecane-α,ω-dicarboxylic acid, dodecane-α,ω-dicarboxylic acid, cis- and trans-cyclohexane-1,2-dicarboxylic acid, cis- and trans-cyclohexane-1,3-dicarboxylic acid, cis- and trans-cyclohexane-1,4-dicarboxylic acid, cis- and trans-cyclopentane-1,2-dicarboxylic acid, cis- and trans-cyclopentane-1,3-dicarboxylic acid, phthalic acid, isophthalic acid, terephthalic acid and mixtures thereof.

The abovementioned dicarboxylic acids can also be substituted. Suitable substituted dicarboxylic acids can exhibit one or more residues preferably chosen from alkyl, cycloalkyl and aryl as defined at the start. Suitable substituted dicarboxylic acids are, for example, 2-methylmalonic acid, 2-ethylmalonic acid, 2-phenylmalonic acid, 2-methylsuccinic acid, 2-ethylsuccinic acid, 2-phenylsuccinic acid, itaconic acid, 3,3-dimethylglutaric acid, and the like.

The dicarboxylic acids can be used either as such or in the form of derivatives. Suitable derivatives are anhydrides and their oligomers and polymers, mono- and diesters, preferably mono- and dialkyl esters, and acid halides, preferably chlorides. Suitable esters are mono- or dimethyl esters, mono- or diethyl esters, and mono- and diesters of higher alcohols, such as, for example, n-propanol, isopropanol, n-butanol, isobutanol, tert-butanol, n-pentanol, n-hexanol, and the like, also mono- and divinyl esters and mixed esters, preferably methyl ethyl ester.

In the context of the present invention, it is also possible to use a mixture of a dicarboxylic acid and one or more of its derivatives. Likewise, it is, in the context of the present invention, possible to use a mixture of several different derivatives of one or more dicarboxylic acids.

Use is made particularly preferably of succinic acid, glutaric acid, adipic acid, phthalic acid, isophthalic acid, terephthalic acid or their mono- or dimethyl esters. Use is made very particularly preferably of adipic acid.

Suitable polyfunctional amines for the preparation of hyperbranched polyamides exhibit 2 or more than 2 (e.g. 3, 4, 5, 6, and the like) primary or secondary amino groups capable of amide formation.

Suitable diamines are straight-chain and branched aliphatic and cycloaliphatic amines with generally approximately 2 to 30, preferably about 2 to 20, carbon atoms. Suitable diamines are, for example, those of the general formula R¹—NH—R²—NH—R³, in which R¹ and R³ represent, independently of one another, hydrogen, alkyl, cycloalkyl or aryl and R² represents alkylene, cycloalkylene or arylene. These include ethylenediamine, 1,2-diaminopropane, 1,3-diaminopropane, 1,4-diaminobutane, 1,5-diaminopentane, 1,6-diaminohexane, 1,7-diaminoheptane, 1,8-diaminooctane, 1,9-diaminononane, 1,10-diaminodecane, 1,11-diaminoundecane, 1,12-diaminododecane, N-alkylethylenediamine, such as N-methylethylenediamine and N-ethylethylenediamine, N,N′-dialkylethylenediamine, such as N,N″-dimethylethylenediamine, N-alkylhexamethylenediamine, such as N-methylhexamethylenediamine, piperazine, bis(4-aminocyclohexyl)methane, phenylenediamine, isophoronediamine, bis(2-aminoethyl)ether, 1,2-bis(2-aminoethoxy)ethane and amine-terminated polyoxyalkylenepolyols (“Jeffamines” or α,ω-diaminopolyethers), which can be prepared, e.g., by amination of polyalkylene oxides with ammonia.

Suitable triamines are, e.g. bis(2-aminoethyl)amine (=diethylenetriamine), N,N′-diethyldiethylenetriamine, bis(3-aminopropyl)amine, bis(6-aminohexyl)amine, 4-aminomethyl-1,8-octamethylenediamine, N′-(3-aminopropyl)-N,N-dimethyl-1,3-propanediamine, melamine, and the like.

Suitable amines of higher valency are N,N′-bis(2-aminoethyl)ethylenediamine (=triethylenetetramine), N,N″-bis(2-aminoethyl)-1,3-diaminopropane, N,N′-bis(3-aminopropyl)-1,4-diaminobutane (=spermine), N,N″-bis(2-aminoethyl)piperazine, N,N″-bis(3-aminopropyl)piperazine, tris(2-aminoethyl)amine, tris(3-aminopropyl)amine, tris(6-aminohexyl)amine, and the like.

Polymeric polyamines are also suitable. These generally exhibit a number-average molecular weight of approximately 400 to 10 000, preferably approximately 500 to 8000. These include, e.g., polyamine with terminal primary or secondary amino groups, polyalkyleneimines, preferably polyethyleneimines, vinylamines obtained by hydrolysis of poly-N-vinylamides, such as, e.g., poly-N-vinylacetamide, the abovementioned α,ω-diamines based on aminated polyalkylene oxides, and copolymers comprising, copolymerized, α,β-ethylenically unsaturated monomers with appropriate functional groups, e.g. aminomethyl acrylate, aminoethyl acrylate, (N-methyl)aminoethyl acrylate, (N-methyl)aminoethyl methacrylate, and the like.

The above-described hyperbranched polyamides can generally already be used as such for the preparation of aqueous formulations of insoluble or only sparingly soluble active substances and effect substances. In an additional embodiment, the above-described hyperbranched polyamides are additionally also subjected to a polymer-analogous reaction, as is described subsequently. Suitable for this are, for example, monocarboxylic acids, monoamines, mono- or polyols, and also mono- and polycarboxylic acids, aminocarboxylic acids, mono- and polyamines, and mono- and polyols with special functional groups for the modification of the properties of the hyperbranched polyamides.

The preparation of the hyperbranched polyamides can be carried out in the presence of a conventional catalyst. These include, e.g., metal oxides and carbonates, strong acids, terephthalates, titanium halides, titanium alkoxides and titanium carboxylates, and the like. Suitable catalysts are disclosed, for example, in U.S. Pat. No. 2,244,192, U.S. Pat. No. 2,669,556, SU 775 106 and U.S. Pat. No. 3,705,881. Additional suitable catalysts are mentioned subsequently with the polyesteramides.

### III) Hyperbranched Polyesteramides

Suitable hyperbranched polyesteramides are disclosed, for example, in WO 99/16810 and WO 00/56804, reference to which is made here in their entirety.

Polyesteramides are very generally polymeric compounds exhibiting ester groups and amide groups. Use may be made, to prepare hyperbranched polyesteramides, in principle of at least divalent compounds chosen from polycarboxylic acids, hydroxycarboxylic acids, aminocarboxylic acids, aminoalcohols, polyamines, polyols and derivatives of the abovementioned compounds. In this connection, first, the condition applies that the compounds are chosen in such a way that the polymers obtained exhibit both ester groups and amide groups. In this connection, in addition, the condition applies that the compounds are chosen in such a way that at least one polyfunctional compound is used which exhibits three or more than three (e.g., 4, 5, 6, and the like) functional groups.

A suitable procedure to prepare hyperbranched polyesteramides starts out from polyfunctional aminoalcohols and polycarboxylic acids, use being made of at least one polyfunctional compound exhibiting three or more than three (e.g., 4, 5, 6, and the like) functional groups.

An additional suitable procedure to prepare hyperbranched polyesteramides starts out from polyfunctional amines, polyfunctional alcohols and polycarboxylic acids, use being made of at least one polyfunctional compound exhibiting three or more than three (e.g., 4, 5, 6, and the like) functional groups.

Suitable polyfunctional aminoalcohols for the preparation of hyperbranched polyester-amides exhibit two or more than two (e.g., 3, 4, 5, 6, and the like) functional groups chosen from hydroxyl groups and primary and secondary amino groups. As defined, aminoalcohols in this connection always exhibit at least one hydroxyl group and at least one primary or secondary amino group. Suitable aminoalcohols are straight-chain and branched aliphatic and cycloaliphatic aminoalcohols with generally 2 to 30, preferably 2 to 20, carbon atoms.

Suitable divalent aminoalcohols are, e.g., 2-aminoethanol (=monoethanolamine), 3-amino-1-propanol, 2-amino-1-propanol, 1-amino-2-propanol, 2-amino-3-phenylpropanol, 2-amino-2-methyl-1-propanol, 2-amino-1-butanol, 4-amino-1-butanol, 2-aminoisobutanol, 2-amino-3-methyl-1-butanol, 2-amino-3,3-dimethylbutanol, 1-amino-1-pentanol, 5-amino-1-pentanol, 2-amino-1-pentanol, 2-amino-4-methyl-1-pentanol, 2-amino-3-methyl-1-pentanol, 2-aminocyclohexanol, 4-aminocyclohexanol, 3-(aminomethyl)-3,5,5-trimethylcyclohexanol, 2-amino-1,2-diphenylethanol, 2-amino-1,1-diphenylethanol, 2-amino-2-phenylethanol, 2-amino-1-phenylethanol, 2-(4-aminophenyl)ethanol, 2-(2-aminophenyl)ethanol, 1-(3-aminophenyl)ethanol, 2-amino-1-hexanol, 6-amino-1-hexanol, 6-amino-2-methyl-2-heptanol, N-methylisopropanolamine, N-ethylisopropanolamine, N-methylethanolamine, 1-ethylaminobutan-2-ol, 4-methyl-4-aminopentan-2-ol, 2-(2-aminoethoxy)ethanol, N-(2-hydroxyethyl)piperazine, 1-amino-2-indanol, N-(2-hydroxyethyl)aniline, amino sugars, such as D-glucosamine, D-galactosamine, 4-amino-4,6-dideoxy-α-D-gluco-pyranose, and mixtures thereof.

Suitable trivalent aminoalcohols and aminoalcohols of higher valency are, e.g., N-(2-hydroxyethyl)ethylenediamine, diethanolamine, dipropanolamine, diisopropanolamine, 2-amino-1,3-propanediol, 3-amino-1,2-propanediol, and the like.

Suitable polycarboxylic acids for the preparation of hyperbranched polyesteramides are those described above for the preparation of hyperbranched polyamides. Reference may be made in their entirety to the suitable and preferred embodiments mentioned therein.

Suitable polyfunctional amines for the preparation of hyperbranched polyesteramides are those described above for the preparation of hyperbranched polyamides. Reference may be made in their entirety to the suitable and preferred embodiments mentioned therein.

Suitable polyfunctional alcohols for the preparation of hyperbranched polyesteramides exhibit two or more than two (e.g., 3, 4, 5, 6, and the like) hydroxyl groups. In this connection, the hydroxyl groups can also be partially or completely replaced by mercapto groups.

Suitable diols are straight-chain and branched aliphatic and cycloaliphatic alcohols with generally approximately 2 to 30, preferably approximately 2 to 20, carbon atoms. These include 1,2-ethanediol, 1,2-propanediol, 1,3-propanediol, 1,2-butanediol, 1,3-butanediol, 1,4-butanediol, 2,3-butanediol, 1,2-pentanediol, 1,3-pentanediol, 1,4-pentanediol, 1,5-pentanediol, 2,3-pentanediol, 2,4-pentanediol 1,2-hexanediol, 1,3-hexanediol, 1,4-hexanediol, 1,5-hexanediol, 1,6-hexanediol, 2,5-hexanediol, 1,2-heptanediol, 1,7-heptanediol, 1,2-octanediol, 1,8-octanediol, 1,2-nonanediol, 1,9-nonanediol, 1,2-decanediol, 1,10-decanediol, 1,12-dodecanediol, 2-methyl-1,3-propanediol, 2-methyl-2-butyl-1,3-propanediol, 2,2-dimethyl-1,3-propanediol, 2,2-dimethyl-1,4-butanediol, pinacol, 2-ethyl-2-butyl-1,3-propanediol, diethylene glycol, triethylene glycol, dipropylene glycol, tripropylene glycol, polyalkylene glycols, cyclopentanediols, cyclohexanediols, and the like.

Suitable triols are, e.g., glycerol, butane-1,2,4-triol, n-pentane-1,2,5-triol, n-pentane-1,3,5-triol, n-hexane-1,2,6-triol, n-hexane-1,2,5-triol, trimethylolpropane and trimethylolbutane. Suitable triols are furthermore the triesters of hydroxycarboxylic acids with trivalent alcohols. Preferably, in this connection, they are triglycerides of hydroxycarboxylic acids, such as, e.g., lactic acid, hydroxystearic acid and ricinoleic acid. Naturally occurring mixtures comprising hydroxycarboxylic acid triglycerides, in particular castor oil, are also suitable. Suitable polyols of higher valency are, e.g., sugar alcohols and their derivatives, such as erythritol, pentaerythritol, dipentaerythritol, threitol, inositol and sorbitol. Reaction products of the polyols with alkylene oxides, such as ethylene oxide and/or propylene oxide, are also suitable. Relatively high molecular weight polyols with a number-average molecular weight in the range of approximately 400 to 6000 g/mol, preferably 500 to 4000 g/mol, can also be used. These include, e.g., polyesterols based on aliphatic, cycloaliphatic and/or aromatic di-, tri- and/or polycarboxylic acids with di-, tri- and/or polyols, and also the polyesterols based on lactone. These furthermore include polyetherols which can be obtained, e.g., by polymerization of cyclic ethers or by reaction of alkylene oxides with an initiator molecule. These furthermore also include conventional polycarbonates with terminal hydroxyl groups known to a person skilled in the art which can be obtained by reaction of the diols described above or also bisphenols, such as bisphenol A, with phosgene or carbonic diesters. α,ω-Polyamidols, poly(methyl (meth)acrylate) α,ω-diols and/or poly(butyl (meth)acrylate α,ω-diols, such as, e.g., MD-1000 and BD-1000 from Goldschmidt, are also suitable.

The preparation of hyperbranched polyesteramides can be carried out according to conventional processes known to a person skilled in the art. In a first embodiment, the preparation of hyperbranched polyesteramides is carried out in a single-stage one-pot process starting from polyfunctional aminoalcohols and dicarboxylic acids, use being made of at least one polyfunctional aminoalcohol exhibiting three or more than three (e.g., 4, 5, 6, and the like) functional groups. The molar ratio of dicarboxylic acid to aminoalcohol preferably lies in a range of 2:1 to 1.1:1, particularly preferably of 1.5:1 to 1.2:1. If, in a suitable embodiment of this single-stage process, only dicarboxylic acids, i.e. monomers of type A₂, and trifunctional aminoalcohols, i.e. monomers of type B₃, are used, it is advisable to interrupt the reaction before the gel point is reached. For the definition of the gel point, see Flory, Principles of Polymer Chemistry, Cornell University Press, 1953, pp. 387-398. The gel point can both be calculated according to the theory of Flory and determined by monitoring the viscosity of the reaction mixture. It is practicable to interrupt the reaction as soon as a rapid rise in the viscosity is observed.

In a second embodiment, the preparation of hyperbranched polyesteramides is carried out in a two-stage one-pot process. In this connection, in the first stage, a prepolymer with free carboxylic acid groups is first prepared and this is subsequently reacted in a second stage with polyfunctional compounds exhibiting functional groups capable of ester or amide formation. In a suitable embodiment, the carboxylic acids A₂ and aminoalcohols B₃ are used for the preparation of the prepolymers in the first stage. The molar ratio of dicarboxylic acid to aminoalcohol preferably lies in a range of 2:1 to 10:1, particularly preferably of 2.5:1 to 5:1 and especially 2.7:1 to 4:1. In this procedure, the gelling of the reaction mixture can generally easily be avoided, even at high reaction rates. Use may be made, for the further reaction of the prepolymers in the second stage, of the abovementioned polyfunctional amines, aminoalcohols and polyamines, if appropriate in combination with additional polycarboxylic acids. Reference is made, for suitable and preferred embodiments of these compounds, to what was said above. Preferably, in the second reaction stage, use is predominantly or exclusively made of divalent compounds in accordance with a chain lengthening.

Comparable polymers to those obtained after the two-stage one-pot process can also be obtained if the hyperbranched polyesteramides obtained after the single-stage one-pot process described above are subjected to a subsequent modification in accordance with a polymer-analogous reaction, it being possible for the above-mentioned polyfunctional amines, alcohols, aminoalcohols and carboxylic acids then to be used for this polymer-analogous reaction. A polymer-analogous reaction, both of the hyperbranched polyesteramides obtained after the single-stage process and of the hyperbranched polyesteramides obtained after the two-stage process, with monofunctional compounds, e.g. monoalcohols, monoamines and monocarboxylic acids, as described more specifically below, is naturally also possible. These monofunctional compounds can exhibit additional functional groups for additional modification of the polymer properties. Suitable stoppers are, for example, fatty acids, fatty acid derivatives, such as anhydrides and esters, fatty alcohols, acids and acid derivatives, which exhibit additional functional groups, and alcohols and amines, which exhibit additional functional groups.

The esterification and amidation reaction for the preparation of hyperbranched polyesteramides, as well as the amidation reaction for the preparation of hyperbranched polyamides, can be carried out in the presence of at least one catalyst. Suitable catalysts are, for example, acidic catalysts, organometallic catalysts, enzymes, and the like.

Suitable acidic catalysts are, e.g., sulfuric acid, phosphoric acid, phosphonic acid, hypophosphorous acid, aluminum sulfate hydrate, alum, acidic silica gel and acidic alumina. Suitable catalysts are furthermore organoaluminum compounds of the general formula Al(OR)₃ and organotitanium compounds of the general formula Ti(OR)₄, the R residues representing, independently of one another, alkyl or cycloalkyl according to the definition given at the start. Preferred R residues are, for example, chosen from isopropyl and 2-ethylhexyl.

Preferred acidic organometallic catalysts are, for example, chosen from dialkyltin oxides of the general formula R₂SnO, R representing, independently of one another, alkyl or cycloalkyl according to the definition given at the start. These preferably include di-n-butyltin oxide, which can be obtained as commercial “Oxo-Tin”.

Suitable acidic organic catalysts are furthermore acidic organic compounds exhibiting at least one acid group chosen from phosphoric acid groups, phosphonic acid groups, sulfoxyl groups, sulfonic acid groups, and the like. p-Toluenesulfonic acid is preferred, for example. Suitable catalysts are furthermore acidic ion-exchange materials, for example polystyrene resins modified with sulfonic acid groups, which are crosslinked in the usual way, e.g. with divinylbenzene.

### IV) Hyperbranched Polyesteramines

In the context of the present invention, the expression “polyesteramines” describes very generally polymeric compounds exhibiting ester groups and amino groups in the chain, amino groups not being part of an amide group. In principle, at least divalent compounds exhibiting one amino group, preferably no longer available for a subsequent reaction, and at least two additional functional groups, capable of an addition or condensation reaction, can be used for the preparation of hyperbranched polyesteramines. These include, for example, N-alkyl-N-(hydroxyalkyl)aminoalkanecarboxylic acids and carboxylic acid derivatives, N,N-di(hydroxyalkyl)aminoalkanecarboxylic acids and carboxylic acid derivatives, N-alkyl-N-(aminoalkyl)aminoalkanecarboxylic acids and carboxylic acid derivatives, N,N-di(aminoalkyl)aminoalkanecarboxylic acids and carboxylic acid derivatives, and the like. In addition to these monomers, the hyperbranched polyesteramines used according to the invention can comprise additional polyfunctional compounds incorporated exhibiting two or more than two (e.g., 3, 4, 5, 6, and the like) functional groups. These include the above-described polycarboxylic acids, polyfunctional amines, polyfunctional alcohols and polyfunctional aminoalcohols, reference to which is made here in their entirety.

The preparation of hyperbranched polyesteramines is preferably carried out with the use of AB₂ and/or AB₃ monomers which can be obtained by a reaction according to the Michael addition type.

In a first embodiment for the preparation of an AB₂ monomer by Michael addition, an aminoalcohol exhibiting a secondary amino group and two hydroxyl groups is reacted with a compound with an activated double bond, e.g. a vinylogous carbonyl compound.

Suitable aminoalcohols exhibiting a secondary amino group and two hydroxyl groups are, e.g., diethanolamine, dipropanolamine, diisopropanolamine, 2-amino-1,3-propanediol, 3-amino-1,2-propanediol, diisobutanolamine, dicyclohexanolamine, and the like.

Suitable compounds with an activated double bond are preferably chosen from esters of α,β-ethylenically unsaturated mono- and dicarboxylic acids with monovalent alcohols. The α,β-ethylenically unsaturated mono- and dicarboxylic acids are preferably chosen from acrylic acid, methacrylic acid, fumaric acid, maleic acid, itaconic acid, crotonic acid, maleic anhydride, monobutyl maleate and mixtures thereof. Preferably, acrylic acid, methacrylic acid and their mixtures are used as acid component. Preferred vinylogous compounds are methyl (meth)acrylate, methyl ethacrylates, ethyl (meth)acrylate, ethyl ethacrylate, n-butyl (meth)acrylate, tert-butyl (meth)acrylate, tert-butyl ethacrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arachidyl (meth)acrylate, behenyl (meth)acrylate, lignoceryl (meth)acrylate, ceryl (meth)acrylate, n-myricyl (meth)acrylate, palmitoleyl (meth)acrylate, oleyl (meth)acrylate, linoleyl (meth)acrylate, linolenyl (meth)acrylate, stearyl (meth)acrylate, lauryl (meth)acrylate and mixtures thereof. Methyl acrylate and n-butyl acrylate are particularly preferred.

In a second embodiment for the preparation of an AB₂ monomer by Michael addition, an aminoalcohol exhibiting a primary amino group and a hydroxyl group is reacted with a compound with an activated double bond.

Suitable aminoalcohols exhibiting a primary amino group and a hydroxyl group are the divalent aminoalcohols mentioned above for the preparation of hyperbranched polyesteramides, reference to which is made here in their entirety. Suitable compounds with activated double bond are those mentioned above in the first embodiment for the preparation of an AB₂ monomer by Michael addition.

In a third embodiment for the preparation of an AB₃ monomer by Michael addition, an aminoalcohol exhibiting a primary amino group, a secondary amino group and a hydroxyl group is reacted with a compound with three activated double bonds.

A suitable aminoalcohol exhibiting a primary amino group, a secondary amino group and a hydroxyl group is hydroxyethylethylenediamine. Suitable compounds with activated double bonds are those mentioned above in the first embodiment for the preparation of an AB₂ monomer by Michael addition.

The reaction according to the Michael addition type is preferably carried out in bulk or in a solvent which is inert under the reaction conditions. Suitable solvents are, e.g., high boiling alcohols, such as glycerol, aromatic hydrocarbons, such as benzene, toluene, xylene, and the like. The reaction is preferably carried out at a temperature in the range of 0 to 100° C., particularly preferably 5 to 80° C. and especially 10 to 70° C. The reaction is preferably carried out in the presence of an inert gas, such as nitrogen, helium or argon, and/or in the presence of a radical inhibitor. General procedures for the addition of aminoalcohols to activated double bonds are known to a person skilled in the art. In a preferred embodiment, the preparation of the monomers by Michael addition and their subsequent reaction in a polycondensation are carried out in the form of a one-pot reaction.

The preparation of the hyperbranched polyesteramines from the abovementioned or from other ABₓ monomers is carried out according to conventional processes known to a person skilled in the art. In a suitable procedure, the preparation of suitable polyesteramines according to the invention is carried out with the use of the above-described AB₂ monomers which can be obtained by Michael addition. These can additionally be reacted in the presence of additional polyfunctional monomers. Suitable polyfunctional monomers are the polyfunctional aminoalcohols, polyfunctional amines, polyfunctional alcohols and polycarboxylic acids mentioned above in the preparation of the hyperbranched polyesteramides, reference to which is made here in their entirety. If desired, hydroxycarboxylic acids can additionally be used as chain extenders. These include, for example, lactic acid, glycolic acid, and the like.

In a suitable embodiment, the preparation of hyperbranched polyesteramines is carried out in the presence of an A₂B₂ monomer. This is preferably chosen from 2-amino-2-ethyl-1,3-propanediol, 2-amino-2-methyl-1,3-propanediol, 1-amino-2,3-propanediol, 2-amino-1,3-propanediol or 2-amino-1-phenyl-1,3-propanediol.

In an additional suitable embodiment, the preparation of the hyperbranched polyesteramines is carried out in the presence of a “core molecule”. Suitable core molecules are, for example, trimethylolpropane, pentaerythritol, alkoxylated polyols, such as ethoxylated trimethylolpropane, ethoxylated glycerol, propoxylated trimethylolpropane or propoxylated glycerol, polyamines, such as tris(2-aminoethyl)amine, ethylenediamine or hexamethylenediamine, diethanolamine, diisopropanolamine, and the like. The addition of the core-forming monomers can be carried out at the beginning or in the course of the reaction.

In an additional suitable embodiment, the preparation of the hyperbranched polyesteramines is carried out with the use of an aromatic AB₂ monomer. Suitable aromatic AB₂ monomers are, e.g., amidol, aminobenzyl alcohol, 2-amino-5-chlorobenzyl alcohol, 2-amino-9-fluorenol, and the like.

The polycondensation reaction for the preparation of hyperbranched polyesteramines can be carried out in the presence of a catalyst. Suitable catalysts are the catalysts described above for the preparation of the hyperbranched polyesteramides, reference to which is made here in their entirety. Enzymes, such as lipases or esterases, are also suitable catalysts. Suitable lipases or esterases can be obtained from Candida cylindracea, Candida lipolytica, Candida rugosa, Candida antartica, Candida utilis, Chromobacterium viscosum, Geotrichum viscosum, Geotrichum candidum, Mucor javanicus, Mucor mihei, pig pancreas, Pseudomonas spp., Pseudomonas fluorescens, Pseudomonas cepacia, Rhizopus arrhizus, Rhizopus delemar, Rhizopus niveus, Rhizopus oryzae, Aspergillus niger, Penicillium roquefortii, Penicillium camembertii, esterases from Bacillus spp. and Bacillus thermoglucosidasius. Preferred enzymes are Candida antartica lipases B and particularly preferably immobilized Candida antartica lipases B, as can be obtained commercially from Novozymes Biotech Inc. under the designation Novozyme 435.

Advantageously, with enzymatic catalysis, the reaction is possible at low temperatures in a range of approximately 40 to 90° C., preferably 60 to 70° C. Preferably, the enzymatic reaction is carried out in the presence of an inert gas, such as carbon dioxide, nitrogen, argon or helium.

The above-described hyperbranched polymers can generally already be used as such for the preparation of aqueous formulations of insoluble or only to a small extent soluble active substances and effect substances. In an additional embodiment, the hyperbranched polymers described above are additionally even subjected to a polymer-analogous reaction. Thus, the polymer properties can, depending on the type and amount of the compounds used for the polymer-analogous reaction, be specifically suitable for the respective application. Suitably modified hyperbranched polymers can be obtained by polymer-analogous reaction of a hyperbranched polymer comprising nitrogen atoms, carrying functional groups capable of a condensation or addition reaction, with at least one compound chosen from
a) compounds which carry at least one functional group complementary to the groups capable of the condensation or addition reaction of the hyperbranched polymer and additionally at least one hydrophilic group,
b) compounds which carry at least one functional group complementary to the groups capable of the condensation or addition reaction of the hyperbranched polymer and additionally at least one hydrophobic group, and mixtures thereof.

In the context of the present invention, “complementary functional groups” is to be understood as a pair of functional groups which can react with one another in a reaction, preferably a condensation or addition reaction. “Complementary compounds” are pairs of compounds which exhibit functional groups complementary to one another.

Preferred complementary functional groups are chosen from the complementary functional groups a and b of the following table, in which R and R′ represent organic groups, such as alkyl, preferably C₁-C₂₀alkyl, such as methyl, ethyl, n-propyl, isopropyl, n-butyl, tert-butyl, the pentyl, hexyl, heptyl or octyl isomers, and the like; cycloalkyl, preferably C₅-C₈ cycloalkyl, especially cyclopentyl and cyclohexyl; aryl, preferably phenyl; heteroaryl, and the like, and in which R′ can also represent hydrogen.

TABLE
Examples of complementary functional groups a) and b)

| Component A | Component B |
|---|---|
| Functional group a) | Functional group b) |
| —SH | —C(O)—OH, —NCO |
| —NH₂ | —C(O)—OR, NCO |
| —OH | —C(O)—O—C(O)—, —NCO, —COOH |
| —NHR | —NCO, —COOH |
| | —NH—C(O)—OR |
| | —NH—CH₂—OH |
| | —NH—CH₂—O—CH₃ |
| | —NH—C(O)—CH(—C(O)OR)(—C(O)—R) |
| | —NH—C(O)—CH(—C(O)OR)₂ |
| | —NH—C(O)—NR′₂ |
| | ═Si(OR)₂ |
| | epoxy |
| —C(O)—OH | epoxy |
| —O—C(O)—CR′═CH₂ | —OH |
| —O—CR′═CH₂ | —NH₂ |
| | —C(O)—CH₂—C(O)—R |
| | —CH═CH₂ |

To form complementary pairs, suitable functional groups are preferably chosen from hydroxyl, primary and secondary amino, thiol, carboxylic acid, carboxylic acid ester, carboxamide, carboxylic acid anhydride, sulfonic acid, sulfonic acid ester, isocyanate, blocked isocyanate, urethane, urea, ether and expoxide groups.

For the reaction, suitable pairs are, for example, on the one hand, compounds with active hydrogen atoms which, e.g., are chosen from compounds with alcohol, primary and secondary amine, and thiol groups and, on the other hand, compounds with correspondingly reactive groups which, e.g., are chosen from carboxylic acid, carboxylic acid ester, carboxamide, carboxylic acid anhydride, isocyanate, urethane, urea, alcohol, ether and epoxide groups. An additional suitable pair comprises, e.g., compounds with epoxide groups, on the one hand, and carboxylic acid groups, on the other hand. In this connection, it is as a rule uncritical which compound of the pair carries the group a) and which the group b).

Hydrophilic compounds are preferably used for the polymer-analogous reaction. Suitable hydrophilic groups are chosen from ionogenic, ionic and nonionic hydrophilic groups. The ionogenic or ionic groups are preferably carboxylic acid groups and/or sulfonic acid groups and/or nitrogen-comprising groups (amines) or carboxylate groups and/or sulfonate groups and/or quaternized or protonated groups. Compounds comprising acid groups can be converted to the corresponding salts by partial or complete neutralization. Suitable bases for the neutralization are, for example, alkali metal bases, such as sodium hydroxide, potassium hydroxide, sodium carbonate, sodium hydrogencarbonate, potassium carbonate or potassium hydrogencarbonate, and alkaline earth metal bases, such as calcium hydroxide, calcium oxide, magnesium hydroxide or magnesium carbonate, and also ammonia and amines, such as trimethylamine, triethylamine, and the like. Charged cationic groups can be produced from compounds with amine nitrogen atoms either by protonation, e.g. with carboxylic acids, such as acetic acid, or by quaternization, e.g. with alkylating agents, such as C₁-C₄ alkyl halides or sulfates. Examples of such alkylating agents are ethyl chloride, ethyl bromide, dimethyl sulfate and diethyl sulfate.

Hyperbranched polymers with ionic hydrophilic groups obtainable by polymer-analogous reaction are water-soluble as a rule.

Preferably, hydroxycarboxylic acids, such as hydroxyacetic acid (glycolic acid), hydroxypropionic acid (lactic acid), hydroxysuccinic acid (malic acid), hydroxypivalic acid, 4-hydroxybenzoic acid, 12-hydroxydodecanoic acid, dimethylolpropionic acid, and the like, are used for the polymer-analogous reaction.

In addition, hydroxysulfonic acids, such as hydroxymethanesulfonic acid or 2-hydroxyethanesulfonic acid, are preferably used for the polymer-analogous reaction.

In addition, mercaptocarboxylic acids, such as mercaptoacetic acid, are preferably used for the polymer-analogous reaction.

In addition, use is preferably made, for the polymer-analogous reaction, of aminosulfonic acids of the formula:

R¹HN—Y—SO₃H

in which:
Y represents o-, m- or p-phenylene or straight-chain or branched C₂-C₆ alkylene optionally substituted by 1, 2 or 3 hydroxyl groups, and
R¹ represents a hydrogen atom, a C₁-C₁₂ alkyl group (preferably a C₁-C₁₀ and in particular a C₁-C₆ alkyl group) or a C₅-C₆ cycloalkyl group, in which the alkyl group or the cycloalkyl group can be optionally substituted by 1, 2 or 3 hydroxyl groups, carboxyl groups or sulfonic acid groups.

The aminosulfonic acids of the above formula are preferably taurine, N-(1,1-dimethyl-2-hydroxyethyl)-3-amino-2-hydroxypropanesulfonic acid or 2-aminoethylaminoethanesulfonic acid.

In addition, use is preferably made, for the polymer-analogous reaction, of α-, β- or γ-amino acids, for example glycine, alanine, valine, leucine, isoleucine, phenylalanine, tyrosine, proline, hydroxyproline, serine, threonine, methionine, cysteine, tryptophan, β-alanine, aspartic acid or glutamic acid.

In addition, use is preferably made, for the polymer-analogous reaction, of polyetherols. Suitable polyetherols are linear or branched substances which exhibit terminal hydroxyl groups, which comprise ether bonds and which exhibit a molecular weight in the range of, e.g., approximately 300 to 10 000. These include, for example, polyalkylene glycols, e.g., polyethylene glycols, polypropylene glycols or polytetrahydrofuran, or copolymers of ethylene oxide, propylene oxide and/or butylene oxide in which the alkylene oxide units are present randomly distributed or copolymerized in the form of blocks. α,ω-Diaminopolyethers, which can be prepared by amination of polyetherols with ammonia, are also suitable. Such compounds are commercially available under the designation Jeffamine®.

In addition, use is made, for the polymer-analogous reaction, of diamines, polyamines and mixtures thereof.

Suitable polyfunctional amines, alcohols and aminoalcohols are those mentioned above.

Suitable hydrophobic groups for the polymer-analogous reaction are preferably chosen from saturated or unsaturated hydrocarbon residues with 8 to 40, preferably 9 to 35, in particular 10 to 30, carbon atoms. They are preferably alkyl, alkenyl, cycloalkyl or aryl residues. The cycloalkyl or aryl residues can exhibit 1, 2 or 3 substituents, preferably alkyl or alkenyl substituents. In the context of the present invention, the term “alkenyl residues” describes residues exhibiting one, two or more carbon-carbon double bonds.

In the context of the present invention, the expression “C₈-C₄₀ alkyl” comprises straight-chain and branched alkyl groups. In this connection, they are preferably straight-chain and branched C₉-C₃₅ alkyl, particularly preferably C₁₀-C₃₀ alkyl and especially C₁₂-C₂₆ alkyl groups. They are preferably, in this connection, predominantly linear alkyl residues, such as those also present in natural or synthetic fatty acids and fatty alcohols, as well as oxo alcohols. These include in particular n-octyl, ethylhexyl, 1,1,3,3-tetramethylbutyl, n-nonyl, n-decyl, n-undecyl, n-dodecyl, n-tridecyl, myristyl, pentadecyl, palmityl (=cetyl), heptadecyl, octadecyl, nonadecyl, arachidyl, behenyl, lignoceryl, ceryl, myricyl, and the like.

C₈-C₄₀Alkenyl preferably represents straight-chain and branched alkenyl groups which can be monounsaturated, diunsaturated or polyunsaturated. They are preferably C₉-C₃₅, in particular C₁₀-C₃₀ and especially C₁₂-C₂₆ alkenyl groups. These include in particular octenyl, nonenyl, decenyl, undecenyl, dodecenyl, tridecenyl, tetradecenyl, pentadecenyl, hexadecenyl, heptadecenyl, octadecenyl, nonadecenyl, linolyl, linoleyl, eleostearyl, and the like, and in particular oleyl (9-octadecenyl).

Preferred compounds for the hydrophobic polymer-analogous reaction are 1-nonylamine, 1-decylamine, 1-undecylamine, 1-undec-10-enylamine, 1-tridecylamine, 1-tetradecylamine, 1-pentadecylamine, 1-hexadecylamine, 1-heptadecylamine, 1-octadecylamine, 1-octadeca-9,12-dienylamine, 1-nonadecylamine, 1-eicosylamine, 1-eicos-9-enylamine, 1-heneicosylamine, 1-docosylamine and in particular oleylamine and 1-hexadecylamine (cetylamine) or amine mixtures prepared from naturally occurring fatty acids, such as, e.g., tallow fatty amines, which predominantly comprise saturated and unsaturated C₁₄-, C₁₆-C₁₈ alkyl amines, or coconut amines, which comprise saturated, monounsaturated and diunsaturated C₈-C₂₂, preferably C₁₂-C₁₄ alkyl amines.

In addition, preference is given to the compound, for the polymer-analogous reaction, chosen from monovalent alcohols exhibiting one of the hydrophobic residues mentioned above. Such alcohols and alcohol mixtures can, e.g., be obtained by hydrogenation of fatty acids from natural fats and oils or of synthetic fatty acids, e.g. from the catalytic oxidation of paraffins. Suitable alcohols and alcohol mixtures can furthermore be obtained by hydroformylation of olefins with simultaneous hydrogenation of the aldehydes, generally resulting in mixtures of straight-chain and branched primary alcohols (oxo alcohols). Suitable alcohols and alcohol mixtures b) can furthermore be obtained by partial oxidation of n-paraffins according to known processes, producing predominantly linear secondary alcohols. The essentially primary, straight-chain and even-numbered Ziegler alcohols obtainable by organoaluminum synthesis are furthermore suitable.

Amines with a primary or secondary amino group, such as, e.g., methylamine, ethylamine, n-propylamine, isopropylamine, dimethylamine, diethylamine, di(n-propyl)amine, diisopropylamine, and the like, are also suitable.

Suitable monovalent alcohols for the polymer-analogous reaction are, e.g., monofunctional alcohols, such as, e.g., methanol, ethanol, n-propanol, isopropanol, octanol, nonanol, decanol, undecanol, dodecanol, tridecanol, tetradecanol, pentadecanol, hexadecanol, heptadecanol, octadecanol, and the like, and mixtures thereof. They can also be monovalent polyetheralcohols with a number-average molecular weight in the range of approximately 500 to 10 000 g/mol, preferably of 1000 to 5000 g/mol. Monovalent polyetheralcohols can be obtained by alkoxylation of monovalent initiator molecules, such as, for example, methanol, ethanol or n-butanol, ethylene oxide or mixtures or ethylene oxide with other alkylene oxides, in particular propylene oxide, being used as alkoxylating agent.

Suitable monoisocyanates for the polymer-analogous reaction are, e.g., C₈-C₄₀ alkyl isocyanates which can be obtained from the abovementioned amines and amine mixtures by phosgenation or from natural or synthetic fatty acids and fatty acid mixtures by the Hofmann reaction, the Curtius rearrangement or the Lossen rearrangement.

The abovementioned compounds for the polymer-analogous reaction can each time by used individually, as mixtures of exclusively hydrophilic compounds or of exclusively hydrophobic compounds, and as mixtures of hydrophilic compounds with hydrophobic compounds. The properties of the hyperbranched polymers can be varied within a broad range by polymer-analogous reaction of hyperbranched polymers, carrying urethane and/or urea groups, with individual compounds or with mixtures thereof.

Some additional embodiments for polymer-analogous reactions are shown below.

Hyperbranched polymers which exhibit polymerizable olefinic groups and which can be used for the preparation of polymers which crosslink under radiation, in particular UV radiation, can be obtained by reaction with compounds comprising acrylate groups, such as, for example, alcohols comprising acrylate groups, such as 2-hydroxyethyl acrylate or 2-hydroxyethyl methacrylate. Epoxide or vinyl ether groups, which can be used for cationically crosslinking polymers, can also be introduced by reaction with appropriately substituted alcohols.

Oxidatively drying hyperbranched polymers can be obtained by reacting polymers comprising NCO or urethane groups with mono- or polyunsaturated fatty acid esters exhibiting at least one OH group or with mono- or polyunsaturated fatty alcohols or fatty amines, in particular with 3 to 40 carbon atoms. For example, esters of linoleic acid, linolenic acid or eleostearic acid comprising OH groups can be reacted with NCO groups. In addition, NCO or urethane groups can, however, also be reacted directly with alcohols or amines comprising vinyl or allyl groups.

For the preparation of hyperbranched polymers exhibiting the various functionalities, it is possible, for example, to allow 2 mol of 2,4-TDI to react with a mixture of 1 mol of trimethylolpropane and 1 mol of dimethylolpropionic acid. In this connection, a product which has both carboxylic acid groups and OH groups is obtained.

In addition, such products can also be obtained by polymerizing with an ABₓ molecule, terminating the polymerization with the desired degree of conversion and subsequently reacting only a portion of the functional groups originally present, for example only a portion of the OH or of the NCO groups. For example, it is thus possible, with an NCO-terminated polymer of 2,4-TDI and glycerol, to react a portion of the NCO groups with ethanolamine and the remaining NCO groups with mercaptoacetic acid.

In addition, an OH-terminated polymer of isophorone diisocyanate and diethanolamine can later be rendered hydrophobic by, for example, reacting a portion of the OH groups with dodecane isocyanate or with dodecanoic acid. Changing the functionality of a hyperbranched polyurethane or adjusting the polymer properties to the application problem can advantageously be carried out immediately after the polymerization reaction without the NCO-terminated polyurethane being isolated beforehand. The functionalization can, however, also be carried out in a separate reaction.

The hyperbranched polymers used according to the invention as a rule exhibit a mean number of functional groups of at least 4. In principle, the number of the functional groups has no upper limit. Generally, the hyperbranched polymers used according to the invention exhibit however, no more than 100 functional groups. Preferably, the hyperbranched polymers exhibit 4 to 30, particularly preferably 5 to 20, functional groups. Preferably, the number-average molecular weight Mₙ lies in a range of 400 to 100 000 g/mol, particularly preferably of 500 to 80 000 g/mol.

The weight-average molecular weight M_{w} preferably lies in a range of 500 to 500 000 g/mol, particularly preferably 1000 to 100 000 g/mol.

The polydispersity (M_{w}/Mₙ) preferably lies in a range of 1.1 to 50, particularly preferably of 1.3 to 45.

The hyperbranched polymers can be used as a mixture or in combination with surface-active substances, such as, e.g., anionic, cationic, zwitterionic or nonionic surfactants or wetting agents. In addition, they can be used in combination with additional polymers, whereby a strengthening of the solubilizing effect may possibly be achieved.

The active substance composition according to the invention can be prepared in various ways.

In a first embodiment of the present invention, the aqueous active substance composition is prepared by first preparing a homogeneous anhydrous mixture comprising hyperbranched polymer and active substance and/or effect substance and subsequently dispersing the mixture thus obtained in water or an aqueous medium. To prepare the homogeneous anhydrous mixture, the active substance will as a rule be incorporated in a liquid form of the hyperbranched polymer composition, for example a melt or, preferably, a solution in an organic solvent. If a solvent is used, the solvent will subsequently be removed as exhaustively as possible and preferably completely, a solid solution of the active substance in the hyperbranched polymer composition being obtained. Suitable solvents for this are in principle those which are capable of dissolving both the active substance and the polymer, for example aliphatic nitriles, such as acetonitrile and propionitrile, N,N-dialkylamides of aliphatic carboxylic acids, such as dimethylformamide and dimethylacetamide, N-alkyllactams, such as N-methylpyrrolidone, the abovementioned aliphatic and alicyclic ethers, for example tetrahydrofuran, halogenated hydrocarbons, such as dichloromethane or dichloroethane, and mixtures of the abovementioned solvents. To prepare the aqueous composition according to the invention, the solid solution thus obtained of the active substance in the hyperbranched polymer composition will subsequently be dispersed in an aqueous medium with stirring. The stirring can be carried out at temperatures in the region of ambient temperature and at elevated temperature, for example at a temperature in the range of 10 to 80° C. and in particular in the range of 20 to 50° C.

In a second embodiment of the present invention, the aqueous active substance composition is prepared by incorporating the active substance and/or effect substance in an aqueous solution/dispersion of the hyperbranched polymer composition. For this, the procedure is such that, as a rule, the incorporation is carried out at a temperature lying above the melting point of the active substance or effect substance and preferably at a temperature at which the active substance or effect substance melt is of low viscosity, i.e. exhibits a viscosity in the range of 1 to 1000 mPa·s (according to DIN 53019-2 at 25° C.). Preferably, the incorporation is carried out with the application of strong shear forces, for example in an UltraTurrax device.

In a third embodiment of the invention, the aqueous active substance composition is prepared by a process comprising the following steps a) to c):
a) preparing a solution of active substance and/or effect substance and, if appropriate, hyperbranched polymer composition in an organic solvent exhibiting a boiling point below that of water, and
b) mixing the solution of the active substance and/or effect substance with water or with an aqueous solution of the hyperbranched polymer, and
c) removing the solvent.

In this connection, it is possible alternatively to proceed in such a way that the solution of the active substance comprises the hyperbranched polymer composition and this solution is mixed with water or that the solution of the active substance comprises only a portion of the hyperbranched polymer composition or no hyperbranched polymer composition and this solution is mixed with an aqueous solution or dispersion of the hyperbranched polymer composition. The mixing can be carried out in suitable stirred vessels, in which either water or the aqueous solution of the hyperbranched polymer composition can be placed and to which the solution of the active substance or effect substance is added or alternatively in which the solution of the active substance or effect substance is placed and to which the water or the aqueous solution of the hyperbranched polymer composition is added. Subsequently, the organic solvent is removed, e.g. by distillation, water being added, if appropriate.

In a preferred alternative form of this embodiment, the active substance solution and the water or the aqueous solution of the hyperbranched polymer composition are added continuously to a mixing region and the mixture is continuously withdrawn from this mixing region, the solvent subsequently being removed from the mixture. The mixing region can be arranged in any way. In principle, any device which makes possible continuous mixing of liquid streams is suitable for this. Such devices are known, e.g., from Continuous Mixing of Fluids (J.-H. Henzler) in Ullmann's Encyclopedia, 5th ed. on CD-Rom, Wiley-VCH. The mixing region can be arranged as static or dynamic mixers or mixed forms thereof. Jet mixers or comparable mixers with nozzles are also in particular suitable as mixing regions. In a preferred embodiment, the mixing region is the device described in “Handbook of Industrial Crystallization” (A. S. Myerson, 1993, Butterworth-Heinemann, page 139, ISBN 0-7506-9155-7) or a comparable device.

The volume ratio of active substance solution to water or aqueous solution of the hyperbranched polymer composition can be varied over a wide range and preferably lies in the range of 10:1 to 1:20 and in particular in the range of 5:1 to 1:10.

Naturally, the solvent should be suitable for dissolving the hyperbranched polymer composition and the active substance in the desired quantitative ratios. The person skilled in the art can determine suitable solvents by routine experiments. Examples of suitable solvents are C₂-C₄ alkanols, such as ethanol, n-propanol, n-butanol or isobutanol, the abovementioned aliphatic and alicyclic ethers, such as diethyl ether, diisopropyl ether, methyl tert-butyl ether, dioxane or tetrahydrofuran, or ketones, such as acetone or methyl ethyl ketone.

In the aqueous active substance compositions according to the invention, it has proved to be advantageous for the weight ratio of active substance and/or effect substance to hyperbranched polymer to lie in the range of 1:10 to 3:1 and in particular in the range of 1:5 to 2:1.

The content of active substance and/or effect substance can be varied over wide ranges. In particular, the hyperbranched polymers used according to the invention make possible the preparation of “active substance concentrates” which comprise the active substance in an amount of at least 5% by weight, based on the total weight of the composition.

The aqueous active substance compositions according to the invention can advantageously be formulated free from solvent or low in solvent, i.e. the proportion of volatile constituents in the aqueous active substance composition is frequently no more than 10% by weight, in particular no more than 5% by weight and especially no more than 1% by weight, based on the total weight of the composition. In this connection, volatile constituents are those exhibiting, at standard pressure, a boiling point of less than 200° C.

The present invention also relates to the solids which can be obtained by drying the aqueous active substance compositions, e.g. powders. The preparation can be carried out according to conventional drying processes known to a person skilled in the art, e.g. by spray drying, drum drying or freeze drying.

A multitude of different active substances and effect substances can be formulated in the aqueous compositions according to the invention. A particular embodiment of the invention relates to the formulation of active substances for plant protection, i.e. of herbicides, fungicides, nematicides, acaricides or insecticides and active substances which regulate plant growth. The present invention consequently also relates to a plant protection composition comprising
A) at least one hyperbranched polymer comprising nitrogen atoms as defined above,
B) at least one active substance for plant protection exhibiting a solubility in water at 25° C. and 1013 mbar of less than 10 g/l, and
C) if appropriate, at least one additional active substance for plant protection other than B) and/or at least one auxiliary.

Examples of fungicidal active substances which can be formulated as aqueous active substance composition according to the invention comprise:
acylalanines, such as benalaxyl, metalaxyl, ofurace or oxadixyl;
amine derivatives, such as aldimorph, dodine, dodemorph, fenpropimorph, fenpropidin, guazatine, iminoctadine, spiroxamine or tridemorph;
anilinopyrimidines, such as pyrimethanil, mepanipyrim or cyprodinil;
antibiotics, such as cycloheximide, griseofulvin, kasugamycin, natamycin, polyoxin and streptomycin;
azoles, such as bitertanol, bromoconazole, cyproconazole, difenoconazole, diniconazole, epoxiconazole, fenbuconazole, fluquinconazole, flusilazole, flutriafol, hexaconazole, imazalil, ipconazole, metconazole, myclobutanil, penconazole, propiconazole, prochloraz, prothioconazole, tebuconazole, tetraconazole, triadimefon, triadimenol, triflumizole or triticonazole;
2-methoxybenzophenones, such as those disclosed in EP-A 897 904 by the general formula (I), e.g. metrafenone;
dicarboximides, such as iprodione, myclozolin, procymidone or vinclozolin;
dithiocarbamates, such as ferbam, nabam, maneb, mancozeb, metam, metiram, propineb, polycarbamate, thiram, ziram or zineb;
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, picobenzamid, probenazole, proquinazid, pyrifenox, pyroquilon, quinoxyfen, silthiofam, thiabendazole, thifluzamide, thiophanate-methyl, tiadinil, tricyclazole or triforine;
nitrophenyl derivatives, such as binapacryl, dinocap, dinobuton or nitrothal-isopropyl;
phenylpyrroles, such as fenpiclonil or fludioxonil;
unclassified fungicides, such as acibenzolar-5-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide;
strobilurins, such as those disclosed in WO 03/075663 by the general formula (I), for example azoxystrobin, dimoxystrobin, fluoxastrobin, kresoxim-methyl, metominostrobin, orysastrobin, picoxystrobin, pyraclostrobin and trifloxystrobin;
sulfenic acid derivatives, such as captafol, captan, dichlofluanid, folpet or tolylfluanid;
cinnamamides and analogous compounds, such as dimethomorph, flumetover or flumorph;
6-aryl-[1,2,4]triazolo[1,5-a]pyrimidines, such as those disclosed, e.g., in WO-98/46608, WO 99/41255 or WO 03/004465, in each case by the general formula (I);
amide fungicides, such as cyflufenamid and (Z)—N—[α-(cyclopropylmethoxyimino)-2,3-difluoro-6-(difluoromethoxy)benzyl]-2-phenylacetamide.

Examples of herbicides which can be formulated as aqueous active substance composition according to the invention comprise:
1,3,4-thiadiazoles, such as buthidazole and cyprazole;
amides, such as allidochlor, benzoylprop-ethyl, bromobutide, chlorthiamid, dimepiperate, dimethenamid, diphenamid, etobenzanid, flamprop-methyl, fosamine, isoxaben, metazachlor, monalide, naptalam, pronamide or propanil;
aminophosphoric acids, such as bilanafos, buminafos, glufosinate-ammonium, glyphosate or sulfosate;
aminotriazoles such as amitrole, or anilides, such as anilofos or mefenacet;
aryloxyalkanoic acid, such as 2,4-D, 2,4-DB, clomeprop, dichlorprop, dichlorprop-P, fenoprop, fluoroxypyr, MCPA, MCPB, mecoprop, mecoprop-P, napropamide, naproanilide or triclopyr;
benzoic acids, such as chloramben or dicamba;
benzothiadiazinones, such as bentazon;
bleachers, such as clomazone, diflufenican, fluorochloridone, flupoxam, fluridone, pyrazolate or sulcotrione;
carbamates, such as carbetamide, chlorbufam, chlorpropham, desmedipham, phenmedipham or vernolate;
quinolinecarboxylic acids, such as quinclorac or quinmerac;
dichloropropionic acids, such as dalapon;
dihydrobenzofurans, such as ethofumesate;
dihydrofuran-3-ones, such as flurtamone;
dinitroanilines, such as benefin, butralin, dinitramine, ethalfluralin, fluchloralin, isopropalin, nitralin, oryzalin, pendimethalin, prodiamine, profluralin, trifluralin;
dinitrophenols, such as bromofenoxim, dinoseb, dinoseb acetate, dinoterb, DNOC or minoterb acetate;
diphenyl ethers, such as acifluorfen-sodium, aclonifen, bifenox, chlornitrofen, difenoxuron, ethoxyfen, fluorodifen, fluoroglycofen-ethyl, fomesafen, furyloxyfen, lactofen, nitrofen, nitrofluorfen or oxyfluorfen;
dipyridyls, such as cyperquat, difenzoquat metilsulfate, diquat or paraquat dichloride;
imidazoles, such as isocarbamid;
imidazolinones, such as imazamethapyr, imazapyr, imazaquin, imazethabenzmethyl, imazethapyr, imazapic or imazamox;
oxadiazoles, such as methazole, oxadiargyl or oxadiazone;
oxiranes, such as tridiphane;
phenols, such as bromoxynil or ioxynil;
phenoxyphenoxypropionic acid esters, such as clodinafop, cyhalofop-butyl, diclofop-methyl, fenoxaprop-ethyl, fenoxaprop-P-ethyl, fenthiaprop-ethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-P-tefuryl;
phenylacetic acids, such as chlorfenac;
phenylpropionic acids, such as chlorphenprop-methyl;
ppi-active substances, (ppi=preplant incorporated), such as benzofenap, flumiclorac-pentyl, flumioxazin, flumipropyn, flupropacil, pyrazoxyfen, sulfentrazone or thidiazimin;
pyrazoles, such as nipyraclofen;
pyridazines, such as chloridazon, maleic hydrazide, norflurazon or pyridate;
pyridinecarboxylic acids, such as clopyralid, dithiopyr, picloram or thiazopyr;
pyrimidyl ethers, such as pyrithiobac acid, pyrithiobac-sodium, KIH-2023 or KIH-6127;
sulfonamides, such as flumetsulam or metosulam;
triazolecarboxamides, such as triazofenamide;
uracils, such as bromacil, lenacil or terbacil;
furthermore benazolin, benfuresate, bensulide, benzofluor, bentazon, butamifos, cafenstrole, chlorthal-dimethyl, cinmethylin, dichlobenil, endothall, fluorbentranil, mefluidide, perfluidone, piperophos, topramezone and prohexadione-calcium;
sulfonylureas, such as amidosulfuron, azimsulfuron, bensulfuron-methyl, chlorimuron-ethyl, chlorsulfuron, cinosulfuron, cyclosulfamuron, ethametsulfuron-methyl, flazasulfuron, halosulfuron-methyl, imazosulfuron, metsulfuron-methyl, nicosulfuron, primisulfuron, prosulfuron, pyrazosulfuron-ethyl, rimsulfuron, sulfometuron-methyl, thifensulfuron-methyl, triasulfuron, tribenuron-methyl, triflusulfuron-methyl or tritosulfuron;
plant protection active substances of the cyclohexenone type, such as alloxydim, clethodim, cloproxydim, cycloxydim, sethoxydim and tralkoxydim. Very particularly preferred herbicidal active substances of the cyclohexenone type are: tepraloxydim (cf. AGROW, No. 243, 11.3.95, page 21, caloxydim) and 2-(1-[2-{4-chlorophenoxy}propyloxyimino]butyl)-3-hydroxy-5-(2H-tetrahydrothiopyran-3-yl)-2-cyclohexen-1-one, and of the sulfonylurea type is: N-(((4-methoxy-6-[trifluoromethyl]-1,3,5-triazin-2-yl)amino)carbonyl)-2-(trifluoromethyl)benzenesulfonamide.

Examples of insecticides which can be formulated as aqueous active substance composition according to the invention comprise:
organophosphates, such as acephate, azinphos-methyl, chlorpyrifos, chlorfenvinphos, diazinon, dichlorvos, dimethylvinphos, dioxabenzofos, dicrotophos, dimethoate, disulfoton, ethion, EPN, fenitrothion, fenthion, isoxathion, malathion, methamidophos, methidathion, methyl parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, pirimiphos-ethyl, pyraclofos, pyridaphenthion, sulprophos, triazophos, trichlorfon, tetrachlorvinphos or vamidothion;
carbamates, such as alanycarb, benfuracarb, bendiocarb, carbaryl, carbofuran, carbosulfan, fenoxycarb, furathiocarb, indoxacarb, methiocarb, methomyl, oxamyl, pirimicarb, propoxur, thiodicarb or triazamate;
pyrethroids, such as bifenthrin, cyfluthrin, cycloprothrin, cypermethrin, deltamethrin, esfenvalerate, etofenprox, fenpropathrin, fenvalerate, cyhalothrin, lambda-cyhalothrin, permethrin, silafluofen, tau-fluvalinate, tefluthrin, tralomethrin, alpha-cypermethrin or zeta-cypermethrin;
arthropodal growth regulators: a) chitin synthesis inhibitors, e.g. benzoylureas, such as chlorfluazuron, diflubenzuron, flucycloxuron, flufenoxuron, hexaflumuron, lufenuron, novaluron, teflubenzuron, triflumuron, buprofezin, diofenolan, hexythiazox, etoxazole or clofentezine; b) ecdysone antagonists, such as halofenozide, methoxyfenozide or tebufenozide; c) juvenile hormones, such as pyriproxyfen, methoprene or fenoxycarb; d) lipid biosynthesis inhibitors such as spirodiclofen;
neonicotinoids, such as flonicamid, clothianidin, dinotefuran, imidacloprid, thiamethoxam, nitenpyram, nithiazine, acetamiprid or thiacloprid;
additional unclassified insecticides, such as abamectin, acequinocyl, acetamiprid, amitraz, azadirachtin, bensultap, bifenazate, cartap, chlorfenapyr, chlordimeform, cyromazine, diafenthiuron, dinotefuran, diofenolan, emamectin, endosulfan, ethiprole, fenazaquin, fipronil, formetanate, formetanate hydrochloride, gamma-HCH, hydramethylnon, imidacloprid, indoxacarb, isoprocarb, metolcarb, pyridaben, pymetrozine, spinosad, tebufenpyrad, thiamethoxam, thiocyclam, XMC and xylylcarb;
N-phenylsemicarbazones, such as those disclosed in EP-A 462 456 by the general formula (I), especially compounds of the general formula (A)

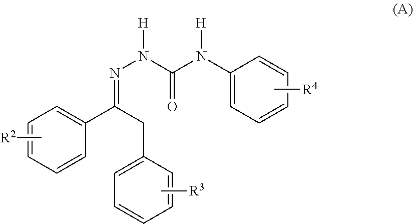

in which R² and R³ represent, independently of one another, hydrogen, halogen, CN, C₁-C₄ alkyl, C₁-C₄ alkoxy, C₁-C₄ haloalkyl or C₁-C₄ haloalkoxy and R⁴ represents C₁-C₄ alkoxy, C₁-C₄ haloalkyl or C₁-C₄ haloalkoxy, e.g. compound IV, in which R² represents 3-CF₃, R³ represents 4-CN and R⁴ represents 4-OCF₃.

Useable growth regulators are, e.g., chlormequat chloride, mepiquat chloride, prohexadione-calcium or the group of the gibberellins. These include, e.g. the gibberellin GA₁, GA₃, GA₄, GA₅ and GA₇, and the like, and the corresponding exo-16,17-dihydrogibberellins, and also the derivatives thereof, e.g. the esters with C₁-C₄ carboxylic acids, The exo-16,17-dihydro-GA₅ 13-acetate is preferred according to the invention.

A preferred embodiment of the invention relates to the use of the hyperbranched polymer compositions according to the invention for the preparation of aqueous active substance compositions of fungicides, in particular strobilurins, azoles and 6-aryltriazolo[1,5-a]pyrimidines, such as those, e.g., disclosed in WO 98/46608, WO-99/41255 or WO 03/004465, in each case by the general formula (I), in particular for active substances of the general formula (B),

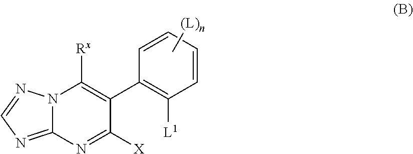

in which:
R^{x} represents an NR⁵R⁶ group, or linear or branched C₁-C₈ alkyl, which is optionally substituted by halogen, OH, C₁-C₄ alkoxy, phenyl or C₃-C₆ cycloalkyl, C₂-C₆ alkenyl, C₃-C₆ cycloalkyl, C₃-C₆ cycloalkenyl, phenyl or naphthyl, it being possible for the last 4 residues mentioned to exhibit 1, 2, 3 or 4 substituents chosen from halogen, OH, C₁-C₄ alkyl, C₁-C₄ haloalkoxy, C₁-C₄ alkoxy and C₁-C₄ haloalkyl;
   R⁵, R⁶ represent, independently of one another, hydrogen, C₁-C₈ alkyl, C₁-C₈ haloalkyl, C₃-C₁₀ cycloalkyl, C₃-C₆ halocycloalkyl, C₂-C₈ alkenyl, C₄-C₁₀ alkadienyl, C₂-C₈ haloalkenyl, C₃-C₆ cycloalkenyl, C₂-C₈ halocycloalkenyl, C₂-C₈ alkynyl, C₂-C₈ haloalkynyl or C₃-C₆ cycloalkynyl, or
      R⁵ and R⁶, together with the nitrogen atom to which they are bonded, form a five- to eight-membered heterocyclyl which is bonded via N and which can comprise one, two or three additional heteroatoms from the group consisting of O, N and S as ring member and/or which can carry one or more substituents from the group consisting of halogen, C₁-C₆ alkyl, C₁-C₆ haloalkyl, C₂-C₆ alkenyl, C₂-C₆ haloalkenyl, C₁-C₆ alkoxy, C₁-C₆ haloalkoxy, C₃-C₆ alkenyloxy, C₃-C₆ haloalkenyloxy, (exo)-C₁-C₆ alkylene and oxy(C₁-C₃ alkylen)oxy;
L is chosen from halogen, cyano, C₁-C₆ alkyl, C₁-C₄ haloalkyl, C₁-C₆ alkoxy, C₁-C₄ haloalkoxy and C₁-C₆ alkoxycarbonyl;
L¹ represents halogen, C₁-C₆ alkyl or C₁-C₆ haloalkyl and in particular fluorine or chlorine;
X represents halogen, C₁-C₄ alkyl, cyano, C₁-C₄ alkoxy or C₁-C₄ haloalkyl and preferably halogen or methyl, and in particular represents chlorine,

Examples of compounds of the formula B are:
5-chloro-7-(4-methylpiperidin-1-yl)-6-(2,4,6-trifluorophenyl)-[1,2,4]triazolo[1,5-a]pyrimidine,
5-chloro-7-(4-methylpiperazin-1-yl)-6-(2,4,6-trifluorophenyl)-[1,2,4]triazolo[1,5-a]pyrimidine,
5-chloro-7-(morpholin-1-yl)-6-(2,4,6-trifluorophenyl)-[1,2,4]triazolo[1,5-a]pyrimidine,
5-chloro-7-(piperidin-1-yl)-6-(2,4,6-trifluorophenyl)-[1,2,4]triazolo[1,5-a]pyrimidine,
5-chloro-7-(morpholin-1-yl)-6-(2,4,6-trifluorophenyl)-[1,2,4]triazolo[1,5-a]pyrimidine,
5-chloro-7-(isopropylamino)-6-(2,4,6-trifluorophenyl)-[1,2,4]triazolo[1,5-a]pyrimidine,
5-chloro-7-(cyclopentylamino)-6-(2,4,6-trifluorophenyl)-[1,2,4]triazolo[1,5-a]pyrimidine,
5-chloro-7-(2,2,2-trifluoroethylamino)-6-(2,4,6-trifluorophenyl)-[1,2,4]triazolo[1,5-a]-pyrimidine,
5-chloro-7-(1,1,1-trifluoroprop-2-ylamino)-6-(2,4,6-trifluorophenyl)-[1,2,4]triazolo[1,5-a]-pyrimidine,
5-chloro-7-(cyclohexylmethyl)-6-(2,4,6-trifluorophenyl)-[1,2,4]triazolo[1,5-a]pyrimidine,
5-chloro-7-(cyclohexyl)-6-(2,4,6-trifluorophenyl)-[1,2,4]triazolo[1,5-a]pyrimidine,
5-chloro-7-(2-methylbut-3-yl)-6-(2,4,6-trifluorophenyl)-[1,2,4]triazolo[1,5-a]pyrimidine,
5-chloro-7-(3-methylprop-1-yl)-6-(2,4,6-trifluorophenyl)-[1,2,4]triazolo[1,5-a]pyrimidine,
5-chloro-7-(4-methylcyclohex-1-yl)-6-(2,4,6-trifluorophenyl)-[1,2,4]triazolo[1,5-a]-pyrimidine,
5-chloro-7-(hex-3-yl)-6-(2,4,6-trifluorophenyl)-[1,2,4]triazolo[1,5-a]pyrimidine,
5-chloro-7-(2-methylbut-1-yl)-6-(2,4,6-trifluorophenyl)-[1,2,4]triazolo[1,5-a]pyrimidine,
5-chloro-7-(3-methylbut-1-yl)-6-(2,4,6-trifluorophenyl)-[1,2,4]triazolo[1,5-a]pyrimidine,
5-chloro-7-(1-methylprop-1-yl)-6-(2,4,6-trifluorophenyl)-[1,2,4]triazolo[1,5-a]pyrimidine,
5-methyl-7-(4-methylpiperidin-1-yl)-6-(2,4,6-trifluorophenyl)-[1,2,4]triazolo[1,5-a]-pyrimidine,
5-methyl-7-(4-methylpiperazin-1-yl)-6-(2,4,6-trifluorophenyl)-[1,2,4]triazolo[1,5-a]-pyrimidine,
5-methyl-7-(morpholin-1-yl)-6-(2,4,6-trifluorophenyl)-[1,2,4]triazolo[1,5-a]pyrimidine,
5-methyl-7-(piperidin-1-yl)-6-(2,4,6-trifluorophenyl)-[1,2,4]triazolo[1,5-a]pyrimidine,
5-methyl-7-(morpholin-1-yl)-6-(2,4,6-trifluorophenyl)-[1,2,4]triazolo[1,5-a]pyrimidine,
5-methyl-7-(isopropylamino)-6-(2,4,6-trifluorophenyl)-[1,2,4]triazolo[1,5-a]pyrimidine,
5-methyl-7-(cyclopentylamino)-6-(2,4,6-trifluorophenyl)-[1,2,4]triazolo[1,5-a]pyrimidine,
5-methyl-7-(2,2,2-trifluoroethylamino)-6-(2,4,6-trifluorophenyl)-[1,2,4]triazolo[1,5-a]-pyrimidine,
5-methyl-7-(1,1,1-trifluoroprop-2-ylamino)-6-(2,4,6-trifluorophenyl)-[1,2,4]triazolo[1,5-a]-pyrimidine,
5-methyl-7-(3,3-dimethylbut-2-ylamino)-6-(2,4,6-trifluorophenyl)-[1,2,4]triazolo[1,5-a]-pyrimidine,
5-methyl-7-(cyclohexylmethyl)-6-(2,4,6-trifluorophenyl)-[1,2,4]triazolo[1,5-a]pyrimidine,
5-methyl-7-(cyclohexyl)-6-(2,4,6-trifluorophenyl)-[1,2,4]triazolo[1,5-a]pyrimidine,
5-methyl-7-(2-methylbut-3-yl)-6-(2,4,6-trifluorophenyl)-[1,2,4]triazolo[1,5-a]pyrimidine,
5-methyl-7-(3-methylprop-1-yl)-6-(2,4,6-trifluorophenyl)-[1,2,4]triazolo[1,5-a]pyrimidine,
5-methyl-7-(4-methylcyclohex-1-yl)-6-(2,4,6-trifluorophenyl)-[1,2,4]triazolo[1,5-a]-pyrimidine,
5-methyl-7-(hex-3-yl)-6-(2,4,6-trifluorophenyl)-[1,2,4]triazolo[1,5-a]pyrimidine,
5-methyl-7-(2-methylbut-1-yl)-6-(2,4,6-trifluorophenyl)-[1,2,4]triazolo[1,5-a]pyrimidine,
5-methyl-7-(3-methylbut-1-yl)-6-(2,4,6-trifluorophenyl)-[1,2,4]triazolo[1,5-a]pyrimidine and 5-methyl-7-(1-methylprop-1-yl)-6-(2,4,6-trifluorophenyl)-[1,2,4]triazolo[1,5-a]-pyrimidine.

An additional preferred embodiment of the invention relates to the use of hyperbranched polymers comprising nitrogen atoms for the preparation of aqueous active substance compositions of insecticides, in particular of arylpyrroles, such as chlorfenapyr, of pyrethroids, such as bifenthrin, cyfluthrin, cycloprothrin, cypermethrin, deltamethrin, esfenvalerate, etofenprox, fenpropathrin, fenvalerate, cyhalothrin, lambda-cyhalothrin, permethrin, silafluofen, tau-fluvalinate, tefluthrin, tralomethrin, alpha-cypermethrin or zeta-cypermethrin, of neonicotinoids and of semicarbazones of the formula A.

Furthermore, the hyperbranched polymers comprising nitrogen atoms to be used according to the invention can be used as solubilizers for UV absorbers which are sparingly soluble or insoluble in water.

The term “UV absorber” comprises, in the context of the present invention, UV-A, UV-B and/or broad broadband filters.

Advantageous broad spectrum screening agents, UV-A filter substances or UV-B filter substances are, for example, representatives of the following classes of compounds:

Bisresorcinyltriazine derivatives with the following structure:

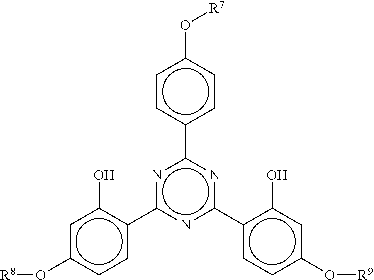

in which R⁷, R⁸ and R⁹ are chosen, independently of one another, from the group of the branched and unbranched alkyl groups with 1 to 10 carbon atoms or represent a single hydrogen atom. Particular preference is given to 2,4-bis[4-(2-ethylhexyloxy)-2-hydroxyphenyl]-6-(4-methoxyphenyl)-1,3,5-triazine (INCI: Anisotriazine), which can be obtained from CIBA Chemikalien GmbH under the trade name Tinosorb® S.

In addition, other UV filter substances exhibiting the structural unit

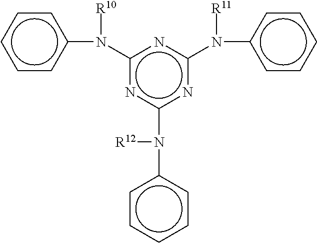

are advantageous UV filter substances in accordance with the invention, for example the s-triazine derivatives disclosed in the European Laid-Open Application EP-570 838 A1, the chemical structure of which is represented by the generic formula

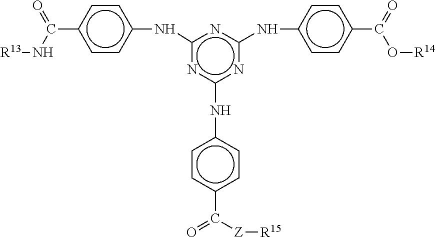

in which
R¹³ represents a branched or unbranched C₁-C₁₈ alkyl residue or a C₈-C₁₂ cycloalkyl residue, optionally substituted with one or more C₁-C₄ alkyl groups,
Z represents an oxygen atom or an NH group,
R¹⁴ represents a branched or unbranched C₁-C₁₈ alkyl residue, a C₅-C₁₂ cycloalkyl residue, optionally substituted with one or more C₁-C₄ alkyl groups, or a hydrogen atom, an alkali metal atom, an ammonium group or a group of the formula

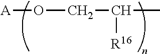

in which
A represents a branched or unbranched C₁-C₁₈ alkyl residue, a C₅-C₁₂ cycloalkyl residue or an aryl residue, optionally substituted with one or more C₁-C₄ alkyl groups,
R¹⁶ represents a hydrogen atom or a methyl group,
n represents a number from 1 to 10,
R¹⁵ represents a branched or unbranched C₁-C₁₈ alkyl residue or a C₅-C₁₂ cycloalkyl residue, optionally substituted with one or more C₁-C₄ alkyl groups, if X represents the NH group, and represents a branched or unbranched C₁-C₁₈ alkyl residue or a C₅-C₁₂ cycloalkyl residue, optionally substituted with one or more C₁-C₄ alkyl groups, or a hydrogen atom, an alkali metal atom, an ammonium group or a group of the formula

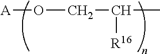

in which
A represents a branched or unbranched C₁-C₁₈ alkyl residue, a C₅-C₁₂ cycloalkyl residue or an aryl residue, optionally substituted with one or more C₁-C₄ alkyl groups,
R¹⁶ represents a hydrogen atom or a methyl group,
n represents a number from 1 to 10,
   if X represents an oxygen atom.

Furthermore, a particularly preferred UV filter substance in accordance with the present invention is an asymmetrically substituted s-triazine, the chemical structure of which is represented by the formula

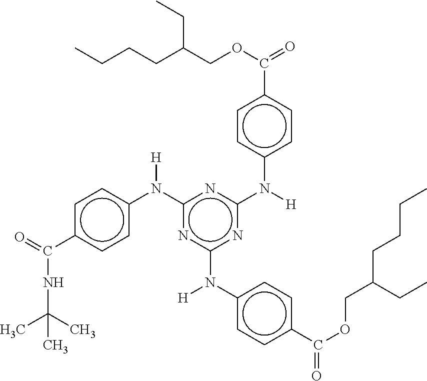

which is also described below as dioctyl butylamido triazone (INCI: Diethylhexyl Butamido Triazone) and can be obtained from Sigma 3V under the trade name Uvasorb® HEB.

Also advantageous in accordance with the present invention is a symmetrically substituted s-triazine, 2,4,6-trianilino-p-(carbo-2′-ethylhexyl-1′-oxy)-1,3,5-triazine (INCI: Ethylhexyl Triazone), which is sold by BASF Aktiengesellschaft under the trade name Uvinul® T 150.

In addition, European Laid-Open Application 775 698 discloses bisresorcinyltriazine derivatives which are preferably to be used, the chemical structure of which is represented by the generic formula

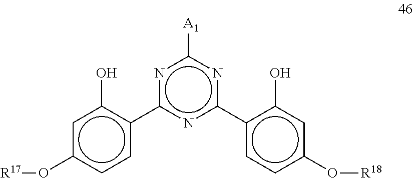

in which R¹⁷ and R¹⁸ represent, inter alia, C₃-C₁₈ alkyl or C₂-C₁₈ alkenyl and A₁ represents an aromatic residue.

The following compounds are advantageous in accordance with the present invention; 2,4-bis{[4-(3-sulfonato)-2-hydroxypropyloxy)-2-hydroxy]phenyl}-6-(4-methoxyphenyl)-1,3,5-triazine, sodium salt, 2,4-bis{[4-(3-(2-propyloxy)-2-hydroxypropyloxy)-2-hydroxy]-phenyl}-6-(4-methoxyphenyl)-1,3,5-triazine, 2,4-bis{[4-(2-ethylhexyloxy)-2-hydroxy]-phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

Advantageous oil-soluble UV-B and/or broadband filters are, e.g.:
3-benzylidenecamphor derivatives, preferably 3-(4-methylbenzylidene)camphor or 3-benzylidenecamphor;
4-aminobenzoic acid derivatives, preferably
4-(dimethylamino)benzoic acid (2-ethylhexyl) ester or
4-(dimethylamino)benzoic acid amyl ester;
   benzophenone derivatives, preferably 2-hydroxy-4-methoxybenzophenone (available from BASF under the trade name Uvinul® M40), 2-hydroxy-4-methoxy-4′-methylbenzophenone, 2,2′-dihydroxy-4-methoxybenzophenone or 2,2′,4,4′-tetrahydroxybenzophenone (available from BASF under the trade name Uvinul® D 50).

Particularly advantageous UV filter substances in the context of the present invention which are liquid at ambient temperature are homomethyl salicylate, 2-ethylhexyl 2-cyano-3,3-diphenylacrylate, 2-ethylhexyl 2-hydroxybenzoate and cinnamic acid esters, preferably 4-methoxycinnamic acid (2-ethylhexyl) ester and 4-methoxycinnamic acid isopentyl ester.

Homomethyl salicylate (INCI: Homosalate) is characterized by the following structure:

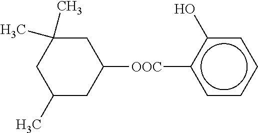

2-Ethylhexyl 2-cyano-3,3-diphenylacrylate (INCI: Octocrylene) is available from BASF under the designation Uvinul® N 539T and is characterized by the following structure:

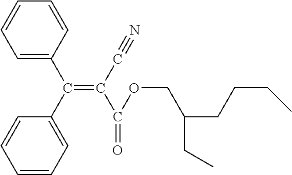 [SMILES: CCCCC(CC)COC(=O)C(C#N)=C(c1ccccc1)c1ccccc1]

2-Ethylhexyl 2-hydroxybenzoate (2-ethylhexyl salicylate, octyl salicylate, INCI: Ethylhexyl Salicylate) is available, for example, from Haarmann & Reimer under the trade name Neo Heliopan® OS and is characterized by the following structure:

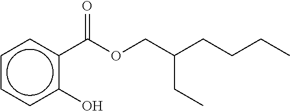

4-Methoxycinnamic acid (2-ethylhexyl) ester (2-ethylhexyl 4-methoxycinnamate, INCI: Ethylhexyl Methoxycinnamate) is, for example, available from BASF under the trade name Uvinul® MC 80 and is characterized by the following structure:

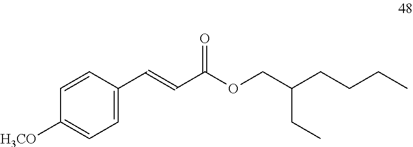 [SMILES: CCCCC(CC)COC(=O)/C=C/c1ccc(OC)cc1]

4-Methoxycinnamic acid isopentyl ester (isopentyl 4-methoxycinnamate, INCI: Isoamyl p-Methoxycinnamate) is, for example, available from Haarmann & Reimer under the trade name Neo Heliopan® E 1000 and is characterized by the following structure:

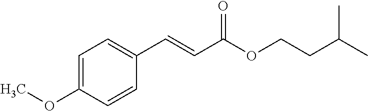 [SMILES: COc1ccc(/C=C/C(=O)OCCC(C)C)cc1]

Advantageous dibenzoylmethane derivatives in accordance with the present invention are, in particular, 4-(tert-butyl)-4′-methoxydibenzoylmethane (CAS No. 70356-09-1), which is sold by BASF under the trade name Uvinul® BMBM and by Merck under the trade name Eusolex® 9020 and which is characterized by the following structure:

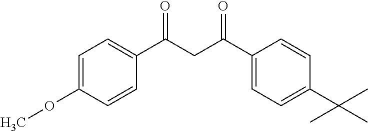 [SMILES: COc1ccc(C(=O)CC(=O)c2ccc(C(C)(C)C)cc2)cc1]

An additional advantageous dibenzoylmethane derivative is 4-isopropyl-dibenzoylmethane (CAS No. 63250-25-9), which is sold by Merck under the name Eusolex® 8020. Eusolex 8020 is characterized by the following structure:

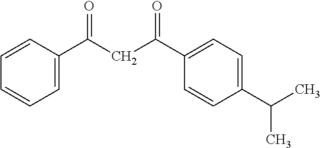 [SMILES: CC(C)c1ccc(C(=O)CC(=O)c2ccccc2)cc1]

Benzotriazoles are characterized by the following structural formula:

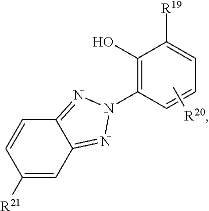

in which

R¹⁹ and R²⁰ represent, independently of one another, linear or branched, saturated or unsaturated, substituted (e.g., substituted with a phenyl residue) or unsubstituted alkyl residues with 1 to 18 carbon atoms.

An advantageous benzotriazole in accordance with the present invention is furthermore 2-(2H-benzotriazol-2-yl)-4-methyl-6-[2-methyl-3-[1,3,3,3-tetramethyl-1-[(trimethyl-silyl)oxy]disiloxanyl]propyl]phenol (CAS No.: 155633-54-8) with the INCI designation Drometrizole Trisiloxane, which is sold by Chimex under the trade name Mexoryl® XL and is characterized by the following structural chemical formula

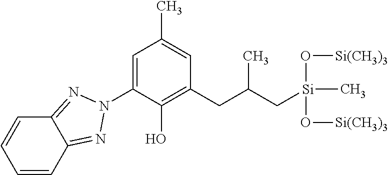

Additional advantageous benzotriazoles in accordance with the present invention are 2,4′-dihydroxy-3-(2H-benzotriazol-2-yl)-5-(1,1,3,3-tetramethylbutyl)-2′-(n-octoxy)-5′-benzoyldiphenylmethane, 2,2′-methylenebis[6-(2H-benzotriazol-2-yl)-4-(methyl)phenol], 2,2′-methylenebis[6-(2H-benzotriazol-2-yl)-4-(1,1,3,3-tetramethylbutyl)phenol], 2-(2′-hydroxy-5′-octylphenyl)benzotriazole, 2-(2′-hydroxy-3′,5′-di(t-amyl)phenyl)benzo-triazole and 2-(2′-hydroxy-5′-methylphenyl)benzotriazole.

An additional UV filter agent advantageous in accordance with the present invention is the diphenylbutadiene compound disclosed in EP-A-0 916 335 of the following formula.

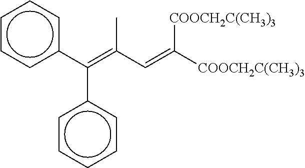

An additional UV-A screening agent which is advantageous in accordance with the present invention is the 2-(4-ethoxyanilinomethylene)propanedicarboxylic acid diethyl ester disclosed in EP-A-0 895 776 of the following formula.

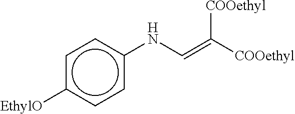

Likewise advantageous in accordance with the present invention is an amino-substituted hydroxybenzophenone of the following formula:

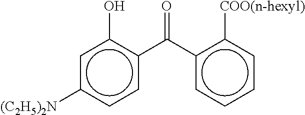

which is sold by BASF Aktiengesellschaft as UV-A screening agent under the trade name Uvinul® A Plus.

The hyperbranched polymers comprising nitrogen atoms used according to the invention are also advantageously suitable as solubilizers for cosmetic compositions. The present invention consequently also relates to a cosmetic composition comprising
A) at least one hyperbranched polymer comprising nitrogen atoms as defined above,
B) at least one cosmetically acceptable active substance or effect substance exhibiting a solubility in water at 25° C. and 1013 mbar of less than 10 g/l, and
C) if appropriate, at least one additional cosmetically acceptable active substance other than B) or auxiliary.

The components B) and C) are preferably chosen, according to their solubility, from cosmetically acceptable carriers, emulsifiers, surfactants, preservatives, perfume oils, thickeners, hair polymers, hair and skin conditioners, water-soluble or dispersible silicone-comprising polymers, bleachers, gelling agents, care agents, colorants, tinting agents, tanning agents, dyes, pigments, antidandruff agents, photofilter agents, deodorizing active substances, vitamins, plant extracts, bodying agents, humectants, refatting agents, collagen, protein hydrolysates, lipids, antioxidants, antifoaming agents, antistatic agents, emollients and softeners.

A comprehensive description of cosmetic auxiliaries is found in H. P. Fiedler, Lexikon der Hilfsstoffe für Pharmazie, Kosmetik und angrenzende Gebiete [Encyclopedia of Auxiliaries for Pharmaceuticals, Cosmetics and Related Fields], 4th edition, Aulendorff: ECV-Editio-Cantor-Verlag, 1996. A comprehensive description of cosmetic raw materials, auxiliaries and active substances, and also suitable formulations, are additionally found in K. Schrader, Grundlagen und Rezepturen der Kosmetika [Fundamental Principles and Formulations of Cosmetics], 2nd edition, Huthig-Verlag, Heidelberg (1989).

Suitable cosmetically acceptable carriers B) which are only water-soluble to a small extent or water-insoluble are, e.g., chosen from oils, fats, waxes, saturated acyclic and cyclic hydrocarbons, fatty acids, fatty alcohols, and the like, and mixtures thereof.

Suitable aqueous carriers C) are, e.g., chosen from water, water-miscible organic solvents, preferably C₁-C₄ alkanols, and mixtures thereof.

The cosmetic compositions according to the invention are solubilizates with a water or water/alcohol base. The solubilizers A) to be used according to the invention are preferably used in the ratio of 0.2:1 to 20:1, preferably 1:1 to 15:1, particularly preferably 2:1 to 12:1, with regard to the sparingly soluble cosmetic active substance or effect substance B).

The content of solubilizer A) to be used according to the invention in the cosmetic compositions preferably lies in the range of 0.01 to 50% by weight, preferably 0.1 to 40% by weight, particularly preferably 1 to 30% by weight, based on the total weight of the composition.

The cosmetic compositions according to the invention exhibit, e.g., an oil or fat component B) chosen from: hydrocarbons of low polarity, such as mineral oils; saturated linear hydrocarbons, preferably with more than 8 carbon atoms, such as tetradecane, hexadecane, octadecane, and the like; cyclic hydrocarbons, such as decahydronaphthalene; branched hydrocarbons; animal and plant oils; waxes; wax esters; petroleum jelly; esters, preferably esters of fatty acids, such as, e.g., the esters of C₁-C₂₄ monoalcohols with C₁-C₂₂ monocarboxylic acids, such as isopropyl isostearate, n-propyl myristate, isopropyl myristate, n-propyl palmitate, isopropyl palmitate, hexacosyl palmitate, octacosyl palmitate, triacontyl palmitate, dotriacontyl palmitate, tetratriacontyl palmitate, hexacosyl stearate, octacosyl stearate, triacontyl stearate, dotriacontyl stearate or tetratriacontyl stearate; salicylates, such as C₁-C₁₀ salicylates, e.g. octyl salicylate; benzoate esters, such as C₁₀-C₁₅ alkyl benzoates or benzyl benzoate; other cosmetic esters, such as fatty acid triglycerides, propylene glycol monolaurate, polyethylene glycol monolaurate, C₁₀-C₁₅ alkyl lactates, and the like; and mixtures thereof.

Suitable silicone oils B) are, e.g. linear polydimethylsiloxanes, poly(methylphenyl)siloxanes, cyclic siloxanes and mixtures thereof. The number-average molecular weight of the polydimethylsiloxanes and poly(methylphenyl)siloxanes preferably lies in a range of approximately 1000 to 150 000 g/mol. Preferred cyclic siloxanes exhibit 4- to 8-membered rings. Suitable cyclic siloxanes are, e.g., commercially available under the designation cyclomethicone.

Preferred oil or fat components B) are chosen from paraffins and paraffin oils; petroleum jelly; natural fats and oils, such as castor oil, soybean oil, peanut oil, olive oil, sunflower oil, sesame oil, avocado oil, cocoa butter, almond oil, persic oil, ricinus oil, cod liver oil, lard, spermaceti, spermaceti oil, sperm oil, wheat germ oil, macadamia nut oil, evening primrose oil or jojoba oil; fatty alcohols, such as lauryl alcohol, myristyl alcohol, cetyl alcohol, stearyl alcohol or oleyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti and mixtures of the abovementioned oil or fat components.

Suitable hydrophilic carriers C) are chosen from water or monovalent, divalent or polyvalent alcohols with preferably 1 to 8 carbon atoms, such as ethanol, n-propanol, isopropanol, propylene glycol, glycerol, sorbitol, and the like.

The cosmetic compositions according to the invention can, e.g., be skin cosmetic, dermatological or hair cosmetic compositions.

The compositions according to the invention are preferably present in the form of a gel, foam, spray, ointment, cream, emulsion, suspension, lotion, milk or paste. If desired, liposomes or microspheres can also be used.

The cosmetically or pharmaceutically active compositions according to the invention can additionally comprise cosmetically and/or dermatologically active substances and auxiliaries.

The cosmetic compositions according to the invention preferably comprise at least one sparingly soluble UV absorber as defined above.

Suitable cosmetically and/or dermatologically active substances are, e.g., coloring active substances, skin and hair pigmentation agents, tinting agents, tanning agents, bleachers, keratin-hardening substances, antimicrobial active substances, photofilter active substances, repellent active substances, substances with a hyperemic activity, substances with a keratolytic and keratoplastic activity, antidandruff active substances, antiinflammatories, substances with a keratinizing activity, substances which act as antioxidants or as radical scavengers, skin moisturizers or humectants, refatting active substances, active substances with an antierythematous or antiallergic activity, and mixtures thereof.

Suitable skin-tanning active substances for artificially tanning the skin without natural or artificial irradiation with UV rays are, e.g., dihydroxyacetone, alloxan and walnut shell extract. Suitable keratin-hardening substances are as a rule active substances as are also used in antiperspirants, such as, e.g., potassium aluminum sulfate, aluminum hydroxychloride, aluminum lactate, and the like. Antimicrobial active substances are used in order to destroy microorganisms or to inhibit their growth and consequently serve both as preservative and as substance with a deodorizing activity which curtails the formation or reduces the intensity of body odors. These include, e.g., conventional preservatives known to a person skilled in the art, such as p-hydroxybenzoic acid esters, imidazolidinylurea, formaldehyde, sorbic acid, benzoic acid, salicylic acid, and the like. Such substances with a deodorizing activity are, e.g., zinc ricinoleate, triclosan, undecylenic acid alkylolamides, triethyl citrate, chlorhexidine, and the like. Suitable photofilter active substances are substances which absorb UV rays in the UV-B and/or UV-A region. Suitable UV filter agents are, e.g., 2,4,6-triaryl-1,3,5-triazines in which the aryl groups can each carry at least one substituent preferably chosen from hydroxyl, alkoxy, especially methoxy, alkoxycarbonyl, especially methoxycarbonyl and ethoxycarbonyl, and mixtures thereof.

Also suitable are cinnamic acid esters, benzophenones, camphor derivatives and pigments which stop UV rays, such as titanium dioxide, talc and zinc oxide. Suitable repellent active substances are compounds which are able to keep off or drive away certain animals, in particular insects, from people. These include, e.g., 2-ethyl-1,3-hexanediol, N,N-diethyl-m-toluamide, and the like. Suitable substances with a hyperemic activity, which stimulate the circulation of blood through the skin, are, e.g., ethereal oils, such as dwarf pine, lavender, rosemary, juniper berry, horse chestnut extract, birch leaf extract, hayseed extract, ethyl acetate, camphor, menthol, peppermint oil, rosemary extract, eucalyptus oil, and the like. Suitable substances with a keratolytic and keratoplastic activity are, e.g., salicylic acid, calcium thioglycolate, thioglycolic acid and its salts, sulfur, and the like. Suitable antidandruff active substances are, e.g., sulfur, sulfur polyethylene glycol sorbitan monooleate, sulfur ricinol polyethoxylate, zinc pyrithione, aluminum pyrithione, and the like. Suitable antiinflammatories, which counteract skin irritations, are, e.g., allantoin, bisabolol, Dragosantol, camomile extract, panthenol, and the like.

The cosmetic compositions can additionally be treated with additional auxiliaries, for example nonionic, cationic or anionic surfactants, such as alkylpolyglycosides, fatty alcohol sulfates, fatty alcohol ether sulfates, alkanesulfonates, fatty alcohol ethoxylates, fatty alcohol phosphates, alkyl betaines, sorbitan esters, POE sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurates, citric acid esters, silicone copolymers, fatty acid polyglycol esters, fatty acid amides, fatty acid alkanolamides, quaternary ammonium compounds, alkylphenol ethoxylates, fatty amine ethoxylates, cosolvents, such as ethylene glycol, propylene glycol, glycerol, inter alia.

Natural or synthetic compounds, e.g. lanolin derivatives, cholesterol derivatives, isopropyl myristate, isopropyl palmitate, electrolytes, dyes, preservatives or acids (e.g., lactic acid or citric acid), can be added as additional constituents.

Suitable cosmetic compositions are, for example, bath additive preparations, such as bath oils, aftershave/preshave lotions, face lotions, mouthwashes, hair lotions, eau de Cologne, eau de toilette and sunscreens.

In the preparation of the solubilizates for cosmetic formulations, the copolymers to be used according to the invention can be introduced neat or, preferably, as an aqueous solution.

Usually, the solubilizer is dissolved in water and vigorously mixed with the sparingly soluble cosmetic active substance to be used each time.

However, the solubilizer can also be vigorously mixed with the sparingly soluble cosmetic active substance to be used each time and subsequently demineralized water can be added with continual stirring.

The present invention consequently also relates to a pharmaceutical composition comprising
A) at least one hyperbranched polymer comprising nitrogen atoms as defined above,
B) at least one pharmaceutically acceptable active substance exhibiting a solubility in water at 25° C. and 1013 mbar of less than 10 g/l, and
C) if appropriate, at least one additional pharmaceutically acceptable active substance other than B) or auxiliary.

The copolymers to be used according to the invention are likewise suitable for use as solubilizer in pharmaceutical preparations of any kind.

The formulation base of the pharmaceutical compositions according to the invention preferably comprises pharmaceutically acceptable auxiliaries. Pharmaceutically acceptable auxiliaries are auxiliaries which are known for use in the field of pharmaceuticals, food technology and related fields, in particular those listed in the relevant pharmacopeias (e.g., DAB, Ph. Eur., BP, NF), and other auxiliaries, the properties of which do not preclude a physiological application.

Suitable auxiliaries can be: lubricants, wetting agents, emulsifying and suspending agents, preservatives, antioxidants, antistimulants, chelating agents, emulsion stabilizers, film-forming agents, gelling agents, odor-masking agents, resins, hydrocolloids, solvents, solubility promoters, neutralizing agents, permeation accelerators, pigments, quaternary ammonium compounds, refatting and superfatting agents, ointment, cream or oil base substances, silicone derivatives, stabilizers, sterilants, propellants, drying agents, opacifiers, thickeners, waxes, softeners or white oils. One embodiment relating to this is based on expert knowledge, as described, for example, in Fiedler, H. P., Lexikon der Hilfsstoffe für Pharmazie, Kosmetik and angrenzende Gebiete [Encyclopedia of Auxiliaries for Pharmaceuticals, Cosmetics and Related Fields], 4th edition, Aulendorf: ECV-Editio-Kantor-Verlag, 1996.

In order to prepare the dermatological compositions according to the invention, the active substances can be mixed or diluted with a suitable auxiliary (excipient). Excipients can be solid, semiliquid or liquid materials which can act as vehicle, carrier or medium for the active substance. The admixing of additional auxiliaries is carried out, if desired, in a way known to a person skilled in the art. It relates in this connection in particular to aqueous solutions or solubilizates for oral or parenteral application. In addition, the copolymers to be used according to the invention are also suitable for use in oral administration forms, such as tablets, capsules, powders or solutions. In this connection, they can make the sparingly soluble pharmaceutical available with increased bioavailability. In the parenteral application, emulsions, for example fatty emulsions, can also be used in addition to solubilizates. The copolymers according to the invention are also suitable for this purpose, in order to process a sparingly soluble pharmaceutical.

Pharmaceutical formulations of the abovementioned kind can be obtained by processing the copolymers to be used according to the invention with pharmaceutical active substances using conventional methods and with the use of known and new active substances.

The use according to the invention can additionally comprise pharmaceutical auxiliaries and/or diluents. Cosolvents, stabilizers and preservatives are especially mentioned as auxiliaries.

The pharmaceutical active substances used are substances which are insoluble or slightly soluble in water. According to DAB 9 (German Pharmacopeia), the solubility of pharmaceutical active substances is categorized as follows: slightly soluble (soluble in 30 to 100 parts of solvent); sparingly soluble (soluble in 100 to 1000 parts of solvent); virtually insoluble (soluble in more than 10 000 parts of solvent). The active substances can in this connection come from any range indicated.

Particular preference is given to those of the abovementioned pharmaceutical compositions relating to formulations which can be applied parenterally.

The content of solubilizer according to the invention in the pharmaceutical compositions lies, depending on the active substance, in the range of 0.01 to 50% by weight, preferably 0.1 to 40% by weight, particularly preferably 1 to 30% by weight, based on the total weight of the composition.

In principle, all pharmaceutical active substances and prodrugs are suitable for the preparation of the pharmaceutical composition according to the invention. These include benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, thyroid therapeutic agents, psychopharmacological agents, antiparkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiasthmatics, broncholytics, beta-receptor blockers, calcium antagonists, ACE inhibitors, antiarteriosclerotics, antiinflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents. Examples of suitable pharmaceutical active substances are in particular the active substances mentioned in paragraphs 0105 to 0131 of US 2003/0157170,

An additional aspect of the present invention relates to the use of the above-mentioned copolymers as solubilizers in molecularly disperse systems. Solid dispersions, that is homogeneous extremely finely disperse phases of two or more solids, and their special case of “solid solutions” (molecularly disperse systems), and their use in pharmaceutical technology, are generally known (cf. Chiou and Riegelmann, J. Pharm. Sci., 1971, 60, 1281-1300). In addition, the present invention also relates to solid solutions comprising at least one copolymer to be used according to the invention.

The preparation of solid solutions can be carried out with the help of fusion processes or according to the solution process.

The copolymers according to the invention are suitable as polymeric auxiliary, i.e. solubilizer for the preparation of such solid dispersions or solid solutions.

According to the fusion process, for example, a sparingly soluble active substance B) and the chosen copolymer A) can be weighed out and mixed in the desired ratio, e.g., in equal parts. A free-falling mixer, for example, is suitable for the mixing. The mixture can subsequently be extruded, e.g. in a twin-screw extruder. The diameter of the cooled product strand thus obtained, consisting of a solid solution of the chosen active substance in the chosen copolymer to be used according to the invention, is dependent on the diameter of the perforation of the perforated plates of the extruder. Cylindrical particles can be obtained by cutting the cooled product strands using a rotating knife, the length of the particles depending on the distance between the perforated plate and the knife. The mean diameter of the cylindrical particle is as a rule approximately 1000 to approximately 3000 μm and the length is as a rule approximately 2000 to approximately 5000 μm. Larger extrudates can be comminuted in an in-line step. Alternatively, a solid solution can also be prepared in the solution process. For this, the chosen sparingly soluble active substance B) and the chosen copolymer A) to be used according to the invention, which acts as solubilizer, are usually dissolved in a suitable solvent. Subsequently, the solution is usually poured into a suitable mold and the solvent is removed, for example by drying. The drying conditions are advantageously chosen according to the properties of the active substance (e.g., thermal lability) and solvent (e.g., boiling point).

Taking into consideration the characteristics of the material, the molded article produced or the extrudate, for example, can be comminuted with a suitable mill (e.g., pin mill). The solid solution is advantageously comminuted down to the mean particle size of less than approximately 2000 μm, preferably less than approximately 1000 μm and particularly preferably less than approximately 500 μm.

The bulk material produced can now be processed, with suitable auxiliaries, to give a tableting mixture or to give a capsule feedstock. The tableting is advantageously carried out so that tablets with a hardness of greater than approximately 35 N, preferably greater than approximately 60 N, particularly preferably approximately 80 to approximately 100 N, are obtained.

Like conventional formulations, the formulations thus obtained can, if necessary, be coated with suitable coating materials in order to obtain resistance to gastric juices, delayed release, masking of taste, and the like.

In addition to use in cosmetics and pharmaceuticals, the copolymers to be used according to the invention are also suitable as solubilizers in the field of foodstuffs for sparingly water-soluble or water-insoluble nutrients, auxiliaries or additives, such as, e.g., fat-soluble vitamins or carotenoids. Mention may be made, as examples, of clear drinks colored with carotenoids. The present invention consequently also relates to food preparations comprising at least one of the copolymers to be used according to the invention as solubilizer. In the context of the present invention, the food preparations are also to be understood as including food supplements, such as, e.g., preparations comprising food dyes, and dietary foods. In addition, the above-mentioned copolymers are also suitable as solubilizers for feed supplements for animal food.

The use of the copolymers to be used according to the invention as solubilizers in agrochemistry can, inter alia, comprise formulations comprising pesticides, herbicides, fungicides or insecticides, above all even those preparations of plant protection agents which are used as spray or pour mixtures.

In addition, the hyperbranched polymers comprising nitrogen atoms used according to the invention are suitable for the preparation of aqueous preparations of food supplements such as water-insoluble vitamins and provitamins, such as vitamin A, vitamin A acetate, vitamin D, vitamin E, tocopherol derivatives, such as tocopherol acetate, and vitamin K.

Examples of effect substances which can be formulated as aqueous active substance composition according to the invention are:

Dyes: e.g., the dyes disclosed in DE-A 10245209 and the compounds described, according to the Colour Index, as disperse dyes and as solvent dyes, which are also described as dispersion dyes. A list of suitable dispersion dyes is found, for example, in Ullmann's Encyclopedia of Industrial Chemistry, 4th edition, Vol. 10, pp. 155-165 (see also Vol. 7, p. 585ff—Anthraquinone Dyes; Vol. 8, p. 244ff—Azo Dyes; Vol. 9, p. 313ff—Quinophthalone Dyes). Particular reference is made herewith to this literature reference and to the compounds mentioned therein. Suitable dispersion dyes and solvent dyes according to the invention comprise the most varied categories of dyes with various chromophores, for example anthraquinone dyes, monoazo and disazo dyes, quinophthalone dyes, methine and azamethine dyes, naphthalimide dyes, naphthoquinone dyes and nitro dyes. Examples of suitable dispersion dyes according to the invention are the dispersion dyes of the following Colour Index list: C. I. Disperse Yellow 1-228, C. I. Disperse Orange 1-148, C. I. Disperse Red 1-349, C. I. Disperse Violet 1-97, C. I. Disperse Blue 1-349, C. I. Disperse Green 1-9, C. I. Disperse Brown 1-21, C. I. Disperse Black 1-36. Examples of suitable solvent dyes according to the invention are the compounds of the following Colour Index list: C. I. Solvent Yellow 2-191, C. I. Solvent Orange 1-113, C. I. Solvent Red 1-248, C. I. Solvent Violet 2-61, C. I. Solvent Blue 2-143, C. I. Solvent Green 1-35, C. I. Solvent Brown 1-63, C. I. Solvent Black 3-50. Suitable dyes according to the invention are furthermore derivatives of naphthalene, of anthracene, of perylene, of terylene or of quarterylene, and diketopyrrolopyrrole dyes, perinone dyes, coumarin dyes, isoindoline and isoindolinone dyes, porphyrin dyes, and phthalocyanine and naphthalocyanine dyes.

In addition to the abovementioned constituents, the active substance and effect substance compositions according to the invention can also comprise conventional surface-active substances and further additives. The surface-active substances include surfactants, dispersing agents and wetting agents. The further additives include in particular thickeners, antifoaming agents, preservatives, antifreeze agents, stabilizers, and the like.

Usable in principle are anionic, cationic, nonionic and amphoteric surfactants, including polymer surfactants and surfactants with heteroatoms in the hydrophobic group.

The anionic surfactants include, for example, carboxylates, in particular alkali metal, alkaline earth metal and ammonium salts of fatty acids, e.g. potassium stearate, which are usually also described as soaps; acyl glutamates; sarcosinates, e.g. sodium lauroyl sarcosinate; taurates; methylcelluloses; alkyl phosphates, in particular mono- and diphosphoric acid alkyl esters; sulfates, in particular alkyl sulfates and alkyl ether sulfates; sulphonates, furthermore alkyl- and alkylarylsulfonates, in particular alkali metal, alkaline earth metal and ammonium salts of arylsulfonic acids and alkyl-substituted arylsulfonic acids, alkylbenzenesulfonic acids, such as, for example, lignin- and phenolsulfonic acid, naphthalene and dibutylnaphthalenesulfonic acids, or dodecylbenzenesulfonates, alkylnaphthalenesulfonates, alkyl methyl ester sulfonates, condensation products of sulfonated naphthalene and derivatives thereof with formaldehyde, condensation products of naphthalenesulfonic acids, phenol- and/or phenolsulfonic acids with formaldehyde or with formaldehyde and urea, or mono- or dialkylsuccinic acid ester sulfonates; and protein hydrolysates and lignosulfite waste liquors. The abovementioned sulfonic acids are advantageously used in the form of their neutral or, if appropriate, basic salts.

The cationic surfactants include, for example, quaternary ammonium compounds, in particular alkyltrimethylammonium and dialkyldimethylammonium halides and alkyl sulfates, and also pyridine and imidazoline derivatives, in particular alkylpyridinium halides.

The nonionic surfactants include, for example:
fatty alcohol polyoxyethylene esters, for example lauryl alcohol polyoxyethylene ether acetate,
alkyl polyoxyethylene and polyoxypropylene ethers, e.g. of isotridecyl alcohol, and fatty alcohol polyoxyethylene ethers,
alkylaryl alcohol polyoxyethylene ethers, e.g. octylphenol polyoxyethylene ether,
alkoxylated animal and/or plant fats and/or oils, for example corn oil ethoxylates, castor oil ethoxylates or tallow fat ethoxylates,
glycerol esters, such as, for example, glycerol monostearate,
fatty alcohol alkoxylates and oxo alcohol alkoxylates, in particular of the RO—(R₁₈O)ᵣ(R₁₉O)ₛR₂₀ type, with R₁₈ and R₁₉, independently of another, ═C₂H₄, C₃H₆ or C₄H₈, R₂₀═H or C₁-C₁₂alkyl, R═C₃-C₃₀ alkyl or C₆-C₃₀ alkenyl, and r and s are, independently of one another, 0 to 50, it not being possible for both to represent 0, such as isotridecyl alcohol and oleyl alcohol polyoxyethylene ether, alkylphenol alkoxylates, such as, for example, ethoxylated isooctyl-, octyl- or nonylphenol, or tributylphenol polyoxyethylene ether,
fatty amine alkoxylates, fatty acid amide alkoxylates and fatty acid diethanolamide alkoxylates, in particular their ethoxylates,
sugar surfactants, sorbitol esters, such as, for example, sorbitan fatty acid esters (sorbitan monooleates or sorbitan tristearate), polyoxyethylene sorbitan fatty acid esters, alkylpolyglycosides or N-alkylgluconamides,
alkyl methyl sulfoxides,
alkyldimethylphosphine oxides, such as, for example, tetradecyldimethyl-phosphine oxide.

Amphoteric surfactants include, for example, sulfobetaines, carboxybetaines and alkyldimethylamine oxides, e.g. tetradecyldimethylamine oxide.

Additional surfactants which should be mentioned here by way of example are perfluorosurfactants, silicone surfactants, phospholipids such as, for example, lecithin or chemically modified lecithins, or amino acid surfactants, e.g. N-lauroyl glutamate.

Unless otherwise specified, the alkyl chains of the abovementioned surfactants are linear or branched residues with usually 8 to 20 carbon atoms.

In one embodiment, the aqueous active substance compositions according to the invention comprise no more than 10% by weight, preferably no more than 5% by weight and in particular no more than 3% by weight, e.g. 0.01 to 5% by weight or 0.1 to 3% by weight, of conventional surface-active substances, each time based on the total amount of active substance and polymer composition. The conventional surface-active substances then preferably make up no more than 5% by weight and in particular no more than 3% by weight, e.g. 0.01 to 5% by weight or 0.1 to 3% by weight, based on the total weight of the composition.

However, depending on the use, it may advantageous for the active substance compositions according to the invention to be formulated with surface-active substances. The proportion of conventional surface-active substance then frequently lies in the range of 0.5 to 30% by weight, in particular in the range of 1 to 20% by weight, based on the total weight of the active substance and polymer composition, or in the range of 0.2 to 20% by weight and in particular in the range of 0.5 to 15% by weight, based on the total weight of the composition formulated.

Even if one advantage of the compositions according to the invention is their low content of volatile organic substances, it may for some applications be desirable for the compositions according to the invention to be used with organic solvents, oils and fats, preferably those solvents or oils and fats which are environmentally friendly or biocompatible, e.g. the abovementioned water-miscible solvents or solvents, oils or fats which are immiscible with water or only miscible with water to a very limited extent, e.g.:
paraffin oils, aromatic hydrocarbons and aromatic hydrocarbon mixtures, e.g. xylenes, Solvesso 100, 150 or 200, and the like,
phenols and alkylphenols, e.g. phenol, hydroquinone, nonylphenol, and the like.
ketones with more than 4 carbon atoms, such as cyclohexanone, isophorone, isopherone, acetophenone or acetonaphthone, alcohols with more than 4 carbon atoms, such as acetylated lanolin alcohol, cetyl alcohol, 1-decanol, 1-heptanol, 1-hexanol, isooctadecanol, isopropyl alcohol, oleyl alcohol or benzyl alcohol,
carboxylic acid esters, e.g. adipic acid dialkyl esters, such as bis(2-ethylhexyl) adipate, phthalic acid dialkyl esters, such as bis(2-ethylhexyl) phthalate, acetic acid alkyl esters (also branched alkyl groups), such as ethyl acetate and ethyl acetoacetate, stearates, such as butyl stearate or glycerol monostearate, citrates, such as tributyl acetylcitrate, in addition cetyl octanoate, methyl oleate, methyl p-hydroxybenzoate, methyl tetradecanoate, propyl p-hydroxybenzoate, methyl benzoate, or lactic acid esters, such as isopropyl lactate, butyl lactate and 2-ethylhexyl lactate,
vegetable oils, such as palm oil, rapeseed oil, ricinus oil and derivatives thereof, such as, e.g. oxidized, coconut oil, cod liver oil, corn oil, soybean oil, linseed oil, olive oil, peanut oil, safflower oil, sesame seed oil, grapefruit oil, basil oil, apricot kernel oil, ginger oil, geranium oil, orange oil, rosemary oil, macadamia oil, onion oil, mandarin oil, pine oil or sunflower oil,
hydrogenated vegetable oils, such as hydrogenated palm oil, hydrogenated rapeseed oil or hydrogenated soybean oil,
animal oils, such as lard oil or fish oils,
dialkylamides of medium- to long-chain fatty acids, e.g. Hallcomides, and
vegetable oil esters, such as rapeseed oil methyl ester.

Suitable thickeners are compounds which bestow a pseudoplastic flow behavior on the formulation, i.e. high viscosity at rest and low viscosity in the agitated state. Mention may be made, in this connection, for example, of polysaccharides or organic layered minerals, such as Xanthan Gum® (Kelzan® from Kelco), Rhodopol® 23 (Rhone-Poulenc) or Veegum® (R.T. Vanderbilt), or Attaclay® (Engelhardt), Xanthan Gum® preferably being used.

Silicone emulsions (such as, e.g., Silicone® SRE, from Wacker, or Rhodorsil® from Rhodia), long-chain alcohols, fatty acids, fluoroorganic compounds and their mixtures, for example, come into consideration as antifoam agents suitable for the dispersions according to the invention.

Bactericides can be added to stabilize the compositions according to the invention against infection by microorganisms. Suitable bactericides are, for example, Proxel® from ICI or Acticide® RS from Thor Chemie and Kathon® MK from Rohm & Haas.

Suitable antifreeze agents are organic polyols, e.g. ethylene glycol, propylene glycol or glycerol. These are generally used in amounts of no more than 10% by weight, based on the total weight of the active substance composition, in order for the desired content of volatile compounds not to be exceeded. In one embodiment of the invention, the proportion therein of the various volatile organic compounds is preferably no more than 1% by weight, in particular no more than 1000 ppm.

If appropriate, the active substance compositions according to the invention can, to regulate the pH, comprise 1 to 5% by weight of buffer, based on the total amounts of the formulation prepared, the amount and the type of the buffer used depending on the chemical properties of the active substance or substances. Examples of buffers are alkali metal salts of weak inorganic or organic acids, such as, e.g., phosphoric acid, boric acid, acetic acid, propionic acid, citric acid, fumaric acid, tartaric acid, oxalic acid and succinic acid.

The following examples of the preparation and use of the hyperbranched polymers to be used according to the invention illustrate the invention without, however, limiting it in any way.

### I. Preparation of Hyperbranched Polymers

### Example 1

### Preparation of a Hyperbranched Polyurea

58.5 g of anhydrous n-butanol were introduced, while flushing with dry nitrogen, into a reaction vessel equipped with a stirrer, an internal thermometer and a nitrogen inlet tube, and the reaction charge was heated to 75° C. 50 g of a polyisocyanate based on the isocyanurate of hexamethylene diisocyanate (Basonat® HI 100, mean NCO functionality approximately 3.7, average molar mass approximately 610 g/mol, BASF Aktiengesellschaft) were then added in 2.5 h so that the temperature of the reaction mixture did not exceed 80° C. After addition of the polyisocyanate, the mixture was stirred at 75° C. for a further 2 h. Subsequently, 0.1 g of potassium hydroxide (dissolved in 1.5 ml of n-butanol), 80.6 g of polyetheramine (Jeffamine® M-1000, monofunctional polyetherol terminated by amino groups, average molar mass approximately 1000 g/mol, Hansman Corp.) and 13.7 g of isophoronediamine were added, and the reaction mixture was stirred at 75° C. for a further 10 min, subsequently heated to 150° C. and stirred at this temperature for a further 3.5 h. The reaction mixture was subsequently allowed to cool down to room temperature, a water-soluble product being obtained. The hyperbranched polyurea was analyzed by gel permeation chromatography with a refractometer as detector. Hexafluoroisopropanol was used as mobile phase and poly(methyl methacrylate) (PMMA) served as standard for determining the molecular weight. In the course of this, a number-average molecular weight Mₙ of 2900 g/mol and a weight-average molecular weight M_{w} of 32 900 g/mol were obtained. On determining the melting point using differential scanning calorimetry (DSC), the product exhibited a melting point of 31.4° C.

### Example 2

### Preparation of a Hyperbranched Polyurea

60 g of tris(aminoethyl)amine, 36.2 g of N,N′-dimethylurea and 0.1 g of potassium carbonate were introduced into a 3-necked flask equipped with a stirrer, a reflux condenser and an internal thermometer, heated to 130° C. and stirred at this temperature for 7.5 h, evolution of gas taking place. After the evolution of gas had subsided, the reaction mixture was heated up to 140° C., stirred for an additional 2 h and then cooled down to ambient temperature. A water-soluble product was obtained and was analyzed as described in example 1: glass transition temperature (Tg): −28° C., Mₙ=3100, M_{w}=5100.

### Example 3

### Preparation of a Hyperbranched Polyamide

1380 g of adipic acid were melted by heating to 150° C. in a 3-necked flask equipped for operating under vacuum. 812 g of diethylenetriamine were added dropwise in one hour into the nitrogen stream at this temperature and left to react further at 130° C. under a reduced pressure of 200 mbar, in order to separate the water formed in the polycondensation. The water formed was collected using a device provided for the azeotropic distillation. As soon as a sharp rise in the viscosity of the reaction mixture was observed, i.e. before reaching the gel point (approximately 4 h), the reaction was halted. A determination of the acid number of the hyperbranched prepolymer according to DIN 53402 resulted in a number of 212 mg KOH/g. 538 g of diethylenetriamine were added to the prepolymer, and the mixture was allowed to react at 130° C. and 200 mbar for a further 8 h. After cooling to ambient temperature, a hyperbranched polyamide was obtained. An analysis as described in example 1 resulted in a number-average molecular weight Mₙ of 3200 g/mol and a weight-average molecular weight M_{w} of 6000 g/mol.

### Example 4

### Preparation of a Hyperbranched Polyesteramide

828 g of diethanolamine and 1380 g of adipic acid were mixed while heating to 130° C. in the nitrogen stream in a round-bottomed flask equipped for operating under inert gas and vacuum, and the mixture was subsequently allowed to react for 2 h in the presence of 2.25 g of dibutyltin oxide as catalyst at 135° C. and under a reduced pressure of 300 mbar, in order to separate the water formed in the polycondensation. As soon as a rapid rise in the viscosity was observed, the acid number (170 mg KOH/g) was determined and 445 g of diethanolamine were added to the prepolymer. After an additional reaction time of 3 h under vacuum at 135° C., a water-soluble hyperbranched polyesteramide was obtained and was analyzed as described in example 1. The product exhibited a number-average molecular weight Mₙ of 3300 g/mol and a weight-average molecular weight M_{w}, of 11 300 g/mol.

### Example 5

### Preparation of a hyperbranched polyesteramine

1. Reaction of butyl acrylate and diethanolamine in the sense of a Michael addition
1744 g of diethanolamine were added dropwise, under a nitrogen atmosphere, to 1800 g of n-butyl acrylate in a 4 l four-necked flask equipped with a device for operating under nitrogen, and the reaction mixture was stirred at ambient temperature for 2 h.

### 2. Polycondensation

7.15 g of dibutyltin oxide were added to the reaction mixture obtained in stage 1 and the mixture was heated to 135° C., the reaction being carried out at a reduced pressure of 200 mbar to separate the methanol formed in the polycondensation reaction. After 25 h, the reaction was halted by cooling the reaction mixture to ambient temperature. The hyperbranched polyesteramine obtained was analyzed as described in example 1. It exhibited a number-average molecular weight Mₙ of 3100 g/mol and a weight-average molecular weight M_{w} of 7600 g/mol.

### Example 6

### Preparation of a Hyperbranched Polyamide

100 g of adipic acid were melted by heating to 150° C. in a 3-necked flask equipped for operation under nitrogen and vacuum. 14 g of diethylenetriamine were then added dropwise into the nitrogen stream in the course of 15 min and the reaction mixture was allowed to react further at 120° C., a reduced pressure of 60 mbar being used to separate the water formed in the polycondensation. The water was collected in a device suitable for the azeotropic distillation. As soon as a strong rise in the viscosity could be observed, i.e. before reaching the gel point (approximately 6 h), the acid number of the prepolymer was determined according to DIN 53402, a number of 521 mg KOH/g being obtained. 95.8 g of diethylenetriamine were added and the reaction mixture was allowed to react at 120° C. and 60 mbar for an additional 10 h. The reaction mixture was subsequently cooled to ambient temperature. The hyperbranched polyamide obtained was analyzed as described in example 1. A number-average molecular weight Mₙ of 4000 g/mol and a weight-average molecular weight M_{w} of 6400 g/mol were obtained.

### II. Performance Properties

### General Procedure 1

Each time 1 or 10% by weight solutions in N,N-dimethylformamide (DMF) of the hyperbranched polymer to be assessed and also 0.1% by weight solutions of the active substances, likewise in DMF, were provided. Active substance compositions were prepared by mixing in a Tecan pipetting robot with the use of 1 ml flat-bottomed glass vessels from HJ-Bioanalytik (96-well plates in the deep well format). Each time 50 μl of polymer and 500 μl of active substance solution were added to a vessel and the solvent was subsequently removed by drying for 24 hours in a vacuum-drying chamber at 70° C. and a pressure of less than 10 mbar. The test samples were subsequently redispersed by addition of 500 μl of buffer solution (phosphate buffer pH 6.8, 23.05 g of potassium dihydrogenphosphate, 23.30 g of disodium hydrogenphosphate, deionized water made up to 5000 ml) and subsequent shaking for two hours using an HP MTP shaker. The assessment was carried out by measuring the particle size using diffusion light scattering after a resting phase of 2 h.

### Assessment:

1=unsatisfactory redispersing, sediment
2=no ambiguous assessment possible
3=complete redispersing with clouding (weak to opaque)
4=clear solution

### General Procedure 2:

0.5 g of the chosen polymer and 0.1 g of a compound to be dissolved in water were dissolved in approximately 20 ml of N,N-dimethylformamide (DMF). The mixture was stirred and subsequently freed from DMF. A solid dispersion of the chosen copolymer with the chosen compound to be dissolved was obtained. The solid dispersion was added to 100 ml of water (buffered to pH 6.8) and the mixture was stirred for 24 h. After filtration, solutions were obtained and their contents of the compound to be dissolved were determined using HPLC and a UV detector. The literature values for water solubilities of the chosen compounds and the wavelengths of the measurement by UV spectroscopy are listed in table 1:

TABLE 1

| Compound to be | Water solubility | Wavelength of the UV |
|---|---|---|
| dissolved | (without polymer) [mg/l] | measurement |
| Uvinul ® T 150 | 0.007 | 308 nm |
| Cinidon-ethyl | 0.057 | 228 nm |
| Pyrene | 0.13 | 330 nm |
| Cl Solvent Red ® | <0.001 | 570 nm |
| Carbamazepine | 120 | 286 nm |
| Piroxicam | 200 | 270 nm |

### General Procedure 3:

Approximately 2 g of polymer were weighed out in a glass beaker. Subsequently, 0.2 g of piroxicam or 0.3 g of carbamazepine was each time weighed out into the charge in order to obtain a supersaturated solution. Subsequently, 20 g of phosphate buffer pH 7.0 were added. After filtration, solutions were obtained and their contents of the compound to be dissolved were determined by UV spectroscopy.

The literature values for water solubilities of the chosen compounds and the wavelengths of the measurement by UV spectroscopy are listed in table 2.

TABLE 2

| Compound to be | Water solubility | Wavelength of the UV |
|---|---|---|
| dissolved | (without polymer) [mg/l] | measurement |
| Carbamazepine | 140 | 286 |
| Piroxicam | 420 | 356 |

### Example 7

Determination of the properties of aqueous active substance compositions according to general procedure 1. Use was made of bentazon at a polymer/active substance ratio of 1:1 and metazachlor at a polymer/active substance ratio of 10:1. The results are listed in table 3.

TABLE 3

| Polymer from example No. | Compound to be dissolved | Assessment |
|---|---|---|
| 1 | Bentazon | 4 |
| 1 | Metazachlor | 3 |
| 6 | Bentazon | 4 |
| 6 | Metazachlor | 3 |

### Example 8

Determination of the properties of aqueous active substance compositions according to general procedure 2. The results are listed in table 4.

TABLE 4

| Compound to be | Solubility [mg/l] in the presence of | | |
|---|---|---|---|
| dissolved | Polymer 1 | Polymer 4 | Polymer 5 |
| Uvinul ® T 150 | 122 | nd | nd |
| Cinidon-ethyl | 97 | nd | nd |
| Cl Solvent Red ® | 30 | nd | nd |
| Fipronil | 448 | nd | nd |
| Carbamazepine | 181 | 336 | 190 |
| Piroxicam | 1020 | nd | 1110 |
| Pyrene | 63 | nd | nd |

### Example 9

Determination of the properties of aqueous active substance compositions according to general procedure 3. The results are listed in table 5.

TABLE 5

| | |
|---|---|
| Compound to be | Solubility [mg/l] in the presence of |

| dissolved | Polymer 1 | Polymer 3 |
|---|---|---|
| Piroxicam | 9200 | 5300 |

1-15. (canceled)

16. A method for solubilizing a substance exhibiting a solubility in water at 25° C. and 1013 mbar of less than 10 g/l, the method comprising:
combining the substance with at least one hyperbranched polymer and water, the at least one hyperbranched polymer selected from the group consisting of molecularly and structurally nonuniform polyurethanes, polyureas, polyamides, polyesteramides and blends thereof, having a degree of branching corresponding to an average number of dendritic linkages and terminal units per molecule of 10 to 90%.

17. The method according to claim 16, comprising a continuous phase which comprises an aqueous medium, and wherein the substance is solubilized or dispersed in the continuous phase.

18. The method according to claim 17, wherein the substance is present in the form of aggregates or particles having a mean particle size which does not exceed a value of 300 nm as determined by dynamic light scattering.

19. The method according to claim 16, wherein the substance and the at least one hyperbranched polymer are present in a ratio by weight of 1:10 to 3:1.

20. The method according to claim 16, wherein the composition has a volatile organic compound content of less than 10% by weight, based on the total weight of the composition.

21. The method according to claim 16, wherein the substance comprises a cosmetically acceptable active substance.

22. The method according to claim 16, wherein the substance comprises a pharmaceutically acceptable active substance.

23. The method according to claim 16, wherein the substance comprises a plant protection active substance.

24. The method according to claim 16, wherein the substance and the at least one hyperbranched polymer are combined to form an anhydrous mixture, and the anhydrous mixture is combined with water.

25. The method according to claim 16, wherein the substance and the at least one hyperbranched polymer are combined in an organic solvent exhibiting a boiling point below that of water, the organic solvent containing the at least one substance and the at least one hyperbranched polymer is combined with water, and the organic solvent is removed.

26. The method according to claim 16, wherein the substance is combined with an organic solvent exhibiting a boiling point below that of water, the organic solvent containing the at least one substance is combined with an aqueous medium containing the at least one hyperbranched polymer, and the organic solvent is removed.

27. The method according to claim 16, wherein the substance is combined with an aqueous medium containing the at least one hyperbranched polymer at a temperature above the melting point of the active substance.

28. The method according to claim 16, wherein the at least one hyperbranched polymer is a hyperbranched polyamide and the preparation of the hyperbranched polyamide starts from polyfunctional amines and polycarboxylic acids, use being made of at least one polyfunctional compound exhibiting three or more than three functional groups, and wherein a first class of monomers with two identical functional groups A₂ is reacted with a second class of monomers Bₙ, and the monomers Bₙ exhibit a mean functionality of at least 2.1.
